SEQUENCE LISTING (5 sequences)

SEQ ID NO: 1 - RNA, 21 nt, Artificial Sequence
  siRNA Green Fluorescent Protein
cgagaagcgcgaucacauguu

SEQ ID NO: 2 - RNA, 23 nt, Artificial Sequence
  siRNA Apolipoprotein B sense strand
gucaucacacugaauaccaauuu

SEQ ID NO: 3 - RNA, 23 nt, Artificial Sequence
  siRNA Apolipoprotein B antisense strand
auugguauucagugugaugacuu

SEQ ID NO: 4 - DNA, 20 bp, Artificial Sequence
  Apolipoprotein B forward primer
tttgccctcaacctaccaac

SEQ ID NO: 5 - DNA, 20 bp, Artificial Sequence
  Apolipoprotein B reverse primer
tgcgatcttgttggctactg

(19) United States
(12) Patent Application Publication
(10) Pub. No.: US 2008/0312174 A1
(43) Pub. Date: Dec. 18, 2008

(54) WATER SOLUBLE CROSSLINKED POLYMERS

(75) Inventors: Lei Yu, Carlsbad, CA (US); Gang Zhao, Vista, CA (US); Nianchun Ma, Oceanside, CA (US); Xin Zhao, San Diego, CA (US); Jian Liu, San Diego, CA (US); Yasunobu Tanaka, San Diego, CA (US)

Correspondence Address: KNOBBE MARTENS OLSON & BEAR LLP, 2040 MAIN STREET, FOURTEENTH FLOOR, IRVINE, CA, 92614, US

(73) Assignee: Nitto Denko Corporation, Osaka (JP)

(21) Appl. No.: 12/126,721

(22) Filed: May 23, 2008

Related U.S. Application Data
(60) Provisional application No. 60/972,686, filed on Sep. 14, 2007.
(60) Provisional application No. 60/942,127, filed on Jun. 5, 2007.

(51) Int. Cl. A61K 31/7105; C12N 15/88; C12Q 1/68; C12N 5/00; C12M 1/00

(52) U.S. Cl. 514/44; 435/458; 435/6; 435/375; 435/285.1

(57) ABSTRACT

Compositions for siRNA delivery are described which include water soluble degradable crosslinked cationic polymers having a water soluble polyethylene glycol component, a cationic polyethyleneimine component and a degradable unit component. The composition may be used to deliver siRNA to cells, particularly cancer cells. The composition may be applied to a solid surface such as a multiwell plate so that the delivery of siRNA may be carried out on the solid surface.

### RELATED APPLICATIONS

This application claims priority to U.S. Provisional Application 60/972,686, filed Sep. 14, 2007 and U.S. Provisional Application 60/942,127, filed Jun. 5, 2007. Both applications are incorporated herein by reference.

### SEQUENCE LISTING

The present application is being filed along with a Sequence Listing in electronic format. The Sequence Listing is provided as a file entitled NDTCO-068PR2-SequenceListing.TXT, created Sep. 14, 2007, which is 2 Kb in size. The information in the electronic format of the Sequence Listing is incorporated herein by reference in its entirety.

### BACKGROUND OF THE INVENTION

1. Field of the Invention

Embodiments described herein relate to compositions and methods for delivering siRNA into a cell. More specifically, embodiments described herein relate to a plate that is coated with a water soluble degradable crosslinked cationic polymer to deliver siRNA into a cell.

2. Description of the Related Art

A number of techniques are available for delivery of plasmid DNA encoded siRNA into cells. For example, cationic polymers, including poly(L-lysine) (PLL), polyethyleneimine (PEI), chitosan, PAMAM dendrimers, and poly(2-dimethylamino)ethyl methacrylate (pDMAEMA), have been used as gene carriers. Unfortunately, transfection efficiency is typically poor with PLL, and high molecular weight PLL has shown significant toxicity to cells. In some cases, PEI provides efficient gene transfer without the need for endosomolytic or targeting agents (see Boussif O., et al., Proc Natl Acad Sci USA. Aug. 1, 1995, 92(16) 7297-301). A range of polyamidoamine dendrimers have been studied as gene-delivery systems (see Eichman J. D., et al., Pharm. Sci. Technol. Today 2000 July; 3(7):232-245). Unfortunately, both high molecular weight PEI and dendrimers that have been found to provide good transfection efficiency have been reported to be toxic to cells. Plasmid DNA carriers made with degradable cationic polymers have been reported to transfer plasmids into mammalian cells with decreased cytotoxicity (see Lim Y. B., et al., J. Am. Chem. Soc., 123 (10), 2460-2461, 2001).

### SUMMARY OF THE INVENTION

Embodiments described herein are directed to a composition for siRNA delivery. In an embodiment, the composition for siRNA deliver can include a water soluble degradable crosslinked cationic polymer that can include: (a) a recurring backbone polyethylene glycol (PEG) unit, (b) a recurring backbone cationic polyethyleneimine (PEI) unit, and (c) a recurring backbone degradable unit that comprises a side chain lipid group.

Embodiments described herein are directed to a method of making the water soluble degradable crosslinked cationic polymers described herein. In some embodiments, a water soluble degradable crosslinked cationic polymer can be synthesized by dissolving a first reactant comprising recurring ethyleneimine units in an organic solvent to form a dissolved or partially dissolved polymeric reactant; reacting the dissolved or partially dissolved polymeric reactant with a degradable monomeric reactant to form a degradable crosslinked polymer, wherein the degradable monomeric reactant comprises a lipid group; and reacting the degradable crosslinked polymer with a third reactant, wherein the third reactant comprises recurring polyethylene glycol units.

Embodiments described herein are directed to methods of delivering siRNA into a cell which includes the following steps: combining any water soluble degradable crosslinked cationic polymer as described herein with the siRNA to form a mixture; and contacting one or more cells with the mixture. More preferably, the siRNA has 19 to 27 base pairs. In preferred embodiments, the cells are mammalian cells. More preferably, the mammalian cells are cancer cells.

Embodiments described herein relate to a method of treating or reducing the risk of cardiovascular disease that can include administering an siRNA corresponding to at least a portion of a coding region of a lipoprotein gene segment complexed with a water soluble degradable crosslinked cationic polymer as described herein to an individual in need thereof.

Embodiments described herein are directed to a device for transfecting a eukaryotic cell with siRNA that can include a solid surface at least partially affixed with a composition comprising a transfection agent, wherein the transfection reagent is selected from a water soluble degradable crosslinked cationic polymer, cationic polymer, lipopolymer, pegylated cationic polymer, pegylated lipopolymer, cationic lipid, pegylated cationic lipid, and cationic degradable pegylated lipopolymer.

Embodiments described herein relate to a method of determining whether siRNA can enter eukaryotic cells. The method can include one or more of the following steps: (a) providing a device described herein; (b) adding the siRNA to the device such that the siRNA interacts with the transfection reagent; (c) seeding the eukaryotic cells onto the device with sufficient density and under appropriate conditions for introduction of the siRNA into the cells; and (d) detecting whether the siRNA have entered the cells.

Embodiments described herein relate to a method for introducing siRNA into eukaryotic cells that can include the steps of: (a) providing a solid surface at least partially coated with a water soluble degradable crosslinked cationic polymer described herein; (b) adding the siRNA to be introduced into the eukaryotic cells onto the cell surface; and (c) seeding cells on the solid surface at a sufficient density and under appropriate conditions for introduction of siRNA into the eukaryotic cells.

### BRIEF DESCRIPTION OF THE DRAWINGS

FIG. 1 shows a method of synthesizing a portion of a water soluble degradable crosslinked cationic polymer.

FIG. 2 shows percent activity of green fluorescent protein in Hela cells after siRNA transfection. The water soluble degradable crosslinked cationic polymers used in this experiment were as follows: polymer 2 (degradable unit:PEI:PEG (12:1:2)), polymer 3 (degradable unit:PEI:PEG (16:1:2)), polymer 4 (degradable unit:PEI:PEG (17:1:2)), polymer 5 (degradable unit:PEI:PEG (20:1:2)). The controls are PEI1200, Cytopure™, Lipofectamine 2000™, and degradable unit:PEI (5:1), all molar ratios. The ratio of polymer to siRNA is 2:1.

FIG. 3 shows percent activity of green fluorescent protein in B16F0 cells after siRNA transfection. The water soluble degradable crosslinked cationic polymers, controls and polymer/siRNA ratios are as stated in the legend to FIG. 2.

FIG. 4 shows percent cell viability for Hela cells after transfection with siRNA. The water soluble degradable crosslinked cationic polymers, controls and polymer/siRNA ratios are as stated in the legend to FIG. 2.

FIG. 5 shows a bar graph plotting green fluorescence (GFP) activity (%) of Hela cells using starting material polyethylenimine-1,200 Daltons (branched PEI-1.2K, negative control), plasmid delivery reagent Cytopure™ (negative control), Lipofectamine 2000™ (L2K), and polymer 1. The ratio of polymer:siRNA is 5:1. The results show that polymer 1 and Lipofectamine 2000™ provide better coated delivery of siRNA to inhibit gene expression than the other known plasmid delivery agent, Cytopure™. Polymer 1 is a water soluble degradable crosslinked cationic polymer having a molar ratio of degradable unit:PEI:PEG (5:1:2).

FIG. 6 shows a bar graph plotting cell viability (%) of Hela cells using starting material polyethylenimine-1,200 daltons (branched PEI-1.2K, negative control), plasmid delivery reagent Cytopure™ (negative control), Lipofectamine 2000™ (L2K), and polymer 1. Polymer 1 is a water soluble degradable crosslinked cationic polymer having a molar ratio of degradable unit:PEI:PEG (5:1:2). The ratio of polymer:siRNA is 5:1. The results show that polymer 1 and L2K do not display cytotoxicity in this assay.

FIG. 7 shows a bar graph plotting green fluorescence (GFP) activity (%) of Hela cells using starting material polyethylenimine-1,200 daltons (branched PEI-1.2K, negative control), plasmid delivery reagent Cytopure™ (negative control), Lipofectamine 2000™ (L2K), and polymer 1. Polymer 1 is a water soluble degradable crosslinked cationic polymer having a molar ratio of degradable unit:PEI:PEG (5:1:2). The ratio of polymer:siRNA is 10:1. The results show that polymer 1 and L2K provide comparable coated delivery of siRNA to inhibit gene expression than the plasmid delivery agent, Cytopure™.

FIG. 8 shows a bar graph plotting cell viability (%) of Hela cells using starting material polyethylenimine-1,200 daltons (branched PEI-1.2K, negative control), plasmid delivery reagent Cytopure™ (negative control), Lipofectamine 2000™ (L2K), and polymer 1. Polymer 1 is a water soluble degradable crosslinked cationic polymer having a molar ratio of degradable unit:PEI:PEG (5:1:2). The ratio of polymer:siRNA is 10:1. The results show that polymer 1 and L2K do not display cytotoxicity in this assay.

FIG. 9 shows a bar graph plotting green fluorescence (GFP) activity (%) of B16F0 cells using starting material polyethylenimine-1,200 daltons (branched PEI-1.2K, negative control), plasmid delivery reagent Cytopure™ (negative control), Lipofectamine 2000™ (L2K), and polymer 1. Polymer 1 is a water soluble degradable crosslinked cationic polymer having a molar ratio of degradable unit:PEI:PEG (5:1:2). The ratio of polymer:siRNA is 2.5:1. The results show that polymer 1 and L2K provide better coated delivery of siRNA to inhibit gene expression than the plasmid delivery agent, Cytopure™.

FIG. 10 shows a bar graph plotting cell viability (%) of B16F0 cells using starting material polyethylenimine-1,200 daltons (branched PEI-1.2K, negative control), plasmid delivery reagent Cytopure™ (negative control), Lipofectamine 2000™ (L2K), and polymer 1. Polymer 1 is a water soluble degradable crosslinked cationic polymer having a molar ratio of degradable unit:PEI:PEG (5:1:2). The ratio of polymer:siRNA is 2.5:1. The results show that polymer 1 and L2K do not display cytotoxicity in this assay.

FIG. 11 shows a bar graph plotting green fluorescence (GFP) activity (%) of B16F0 cells using starting material polyethylenimine-1,200 daltons (branched PEI-1.2K, negative control), plasmid delivery reagent Cytopure™ (negative control), Lipofectamine 2000™ (L2K), and polymer 1. Polymer 1 is a water soluble degradable crosslinked cationic polymer having a molar ratio of degradable unit:PEI:PEG (5:1:2). The ratio of polymer:siRNA is 5:1. The results show that polymer 1 and L2K provide better coated delivery of siRNA to inhibit gene expression than the plasmid delivery agent, Cytopure™

FIG. 12 shows a bar graph plotting cell viability (%) of B16F0 cells using starting material polyethylenimine-1,200 daltons (branched PEI-1.2K, negative control), plasmid delivery reagent Cytopure™ (negative control), Lipofectamine 2000™ (L2K,), and polymer 1. Polymer 1 is a water soluble degradable crosslinked cationic polymer having a molar ratio of degradable unit:PEI:PEG (5:1:2). The ratio of polymer:siRNA is 5:1. The results show that polymer 1 and L2K do not display cytotoxicity in this assay.

FIG. 13 shows a bar graph plotting green fluorescence (GFP) activity (%) of B16F0 cells using starting material polyethylenimine-1,200 daltons (branched PEI-1.2K, negative control), plasmid delivery reagent Cytopure™ (negative control), Lipofectamine 2000™ (L2K), and polymer 1. Polymer 1 is a water soluble degradable crosslinked cationic polymer having a molar ratio of degradable unit:PEI:PEG (5:1:2). The ratio of polymer:siRNA is 10:1. The results show that polymer 1 and L2K provide better coated delivery of siRNA to inhibit gene expression than the other known plasmid delivery agent, Cytopure™

FIG. 14 shows a bar graph plotting cell viability (%) of B16F0 cells using starting material polyethylenimine-1,200 daltons (branched PEI-1.2K, negative control), plasmid delivery reagent Cytopure™ (negative control), Lipofectamine 2000™ (L2K), and polymer 1. Polymer 1 is a water soluble degradable crosslinked cationic polymer having a molar ratio of degradable unit:PEI:PEG (5:1:2). The ratio of polymer:siRNA is 10:1. The results show that polymer 1 do not display cytotoxicity in this assay.

FIG. 15 shows increasing amount of transfection agent polymer 6/siApo-B complexes versus inhibition of apo-B expression in HepG2 cell culture. Polymer 6 is a water soluble degradable crosslinked cationic polymer where the molar ratio of degradable unit:PEI:PEG is 16.5:1:2. The control treatments included polymer 6 and siApo-B (5 μg) alone.

FIG. 16 shows the stability of the transfection agent/siRNA complexes in 5% glucose by fluorescence using RiboGreen™ integration assay. The transfection agent was polymer 2 and the siRNA was anti-Apo-B. Polymer 2 is described in the legend to FIG. 2.

FIG. 17 shows inhibition of expression of apo-B in nude mice by anti-Apo-B using polymer 6 as transfection agent. Polymer 6 is a water soluble degradable crosslinked cationic polymer where the molar ratio of degradable unit:PEI:PEG is 16.5:1:2. Controls included PBS (A), siApo-B (1 mg/kg) (B), polymer 6 (5 mg/kg) (C), and polymer and random siRNA at a ratio of 5:1, administration of 1 mg/kg siApo-B, at 48 hours post-op (F). Treatments included 1.0 mg/kg anti-Apo-B siRNA at 48 hours post-op (D) and 2.5 mg/kg anti-Apo-B siRNA at 2 weeks post-op (E). The ratio of polymer to siRNA was 5/1.

FIG. 18 shows the effect of varying the ratio of transfection agent (polymer 6) to siRNA (anti-Apo-B) for inhibition of Apo-B in nude mice. Polymer 6 is a water soluble degradable crosslinked cationic polymer where the molar ratio of degradable unit:PEI:PEG is 16.5:1:2. Control is PBS (A). Treatments are polymer 6+siApo-B at a ratio of 5:1 (B), 7.5:1 (C), and 10:1 (D) (weight ratios). In all treatments (B-D) 1 mg/kg siApo-B was administered.

FIG. 19 shows the time course of inhibition of Apo-B mRNA expression after injection of transfection agent, polymer 6:siRNA (anti-Apo-B) complexes, into the tail vein of nude mice. Polymer 6 is a water soluble degradable crosslinked cationic polymer where the molar ratio of degradable unit:PEI:PEG is 16.5:1:2. Control is PBS (A). Treatments are polymer 6+siApo-B administered at 1 mg/kg siApo_B measured 48 hours post-op (B), polymer 6+siApo-B administered at 2.5 mg/kg siApo_B measured 1 week post-op (C), and polymer 6+siApo-B administered at 2.5 mg/kg siApo_ measured 2 weeks post-op (D). In all treatments (B-D) the ratio of polymer to siRNA was 5:1, weight ratio.

FIG. 20 shows the time course of inhibition of Apo-B mRNA expression after injection of transfection agent, polymer 6: siRNA (anti-Apo-B) complexes, into the tail vein of C57BL/6 mice. Polymer 6 is a water soluble degradable crosslinked cationic polymer where the molar ratio of degradable unit:PEI:PEG is 16.5:1:2. The controls include PBS (A) abd siapo-B (1 mg/kg). Treatments include polymer 6+siapo-B (5/1, weight to weight ratio, 1 mg/kg of siapo-B)—48 HOURS(C), polymer 6+siapo-B (5/1, weight to weight ratio, 1 mg/kg of siapo-B)—1 WEEK (D), polymer 6+siapo-B (5/1, weight to weight ratio, 1 mg/kg of siapo-B)—2 WEEKS (E), polymer 6+siapo-B (5/1, weight to weight ratio, 1 mg/kg of siapo-B)—3 WEEKS (F).

The drawings are intended to illustrate certain embodiment described herein and are not intended to limit the invention.

### DETAILED DESCRIPTION OF THE PREFERRED EMBODIMENT

Embodiments described herein are directed to the delivery of siRNA into one or more cells. The siRNA delivery may be carried out in solution, preferably in an aqueous solution or more preferably, on a solid surface such as a transfection device. In preferred embodiments, the methods described herein include water soluble degradable crosslinked cationic polymers as transfection agents which are highly effective in the transport of siRNA into cells.

Embodiments described herein relate to water soluble degradable crosslinked cationic polymers that can include in the backbone of the polymer one or more degradable units comprising a side chain lipid group, one or more cationic polyethyleneimine (PEI) units, and one or more polyethylene glycol (PEG) units.

In some embodiments, the recurring backbone polyethylene glycol unit can have a molecular weight in the range of about 50 Daltons to about 5,000 Daltons. In an embodiment, the recurring backbone polyethylene glycol unit can have a molecular weight in the range of about 400 Daltons to about 600 Daltons.

In some embodiments, the recurring backbone cationic polyethyleneimine unit can have a molecular weight in the range of about 200 Daltons to about 25,000 Daltons. In an embodiment, the recurring backbone cationic polyethyleneimine unit can have a molecular weight in the range of about 600 Daltons to about 2,000 Daltons.

In preferred embodiments, the recurring backbone degradable unit can be a recurring unit of Formula (I):

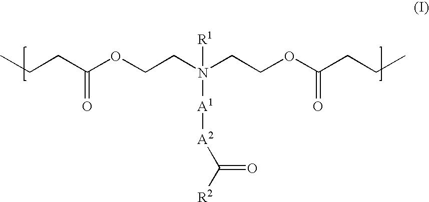

In Formula (I), A¹ can be absent or an optionally substituted substituent selected from the group consisting of: alkyl, alkenyl, alkynyl, heteroalkyl, heteroalkenyl, heteroalkynyl and —(CH₂)ₙ₁-D-(CH₂)ₙ₂—; wherein n1 and n2 can be each independently 0 or an integer in the range of 1 to 10; and D can be an optionally substituted substituent selected from the group consisting of cycloalkyl, cycloalkenyl, cycloalkynyl, aryl, heteroaryl and heterocyclyl; A² can be absent, an oxygen atom or —N(R^{N}), wherein R^{N} is H or C₁₋₆ alkyl; R¹ can be an electron pair, hydrogen, or an optionally substituted substituent selected from the group consisting of alkyl, alkenyl, alkynyl, heteroalkyl, heteroalkenyl, heteroalkynyl, aryl, heteroaryl, and heterocyclyl, wherein if R¹ is hydrogen, or an optionally substituted substituent selected from the group consisting of alkyl, alkenyl, alkynyl, heteroalkyl, heteroalkenyl, heteroalkynyl, aryl, heteroaryl, and heterocyclyl, then the nitrogen atom to which R¹ is attached has an associated positive charge; and R² can be selected from the group consisting of C₂-C₅₀ alkyl, C₂-C₅₀ heteroalkyl, C₂-C₅₀ alkenyl, C₂-C₅₀ heteroalkenyl, C₂-C₅₀ alkynyl, C₂-C₅₀ heteroalkynyl, C₅-C₅₀ aryl, C₅-C₅₀ heteroaryl, —(CH₂)ₚ₁-E-(CH₂)ₚ₂—, and sterol; wherein p1 and p2 can be each independently 0 or an integer in the range of 1 to 40; and E can be an optionally substituted substituent selected from the group consisting of cycloalkyl, cycloalkenyl, cycloalkynyl, aryl, heteroaryl and heterocyclyl. In an embodiment, R² can be C₄-C₃₀ alkyl, C₄-C₃₀ alkenyl, C₄-C₃₀ alkynyl or a sterol. In preferred embodiments, R² can be C₈-C₂₄ alkyl, C₈-C₂₄ alkenyl, C₈-C₂₄ alkynyl or a sterol. While not wanting to be bound by theory, it is believed that the ester groups in Formula (I) impart improved biodegradability to the water soluble degradable crosslinked cationic polymer.

In some embodiments, R² can be a lipid group. In some embodiments, R² can be selected from the group consisting of oleyl, lauryl, myristyl, palmityl, margaryl, stearyl, arachidyl, behenyl and lignoceryl. In an embodiment, R² can be oleyl. In some embodiments, R² can be a sterol. In an embodiment, the sterol can be a cholesteryl moiety.

The nitrogen atom to which R¹ is attached in Formula (I) can have an electron pair, a hydrogen, or an optionally substituted substituent selected from the group consisting of alkyl, alkenyl, alkynyl, heteroalkyl, heteroalkenyl, heteroalkynyl, aryl, heteroaryl, and heterocyclyl bonded to it. Those skilled in the art understand that when the nitrogen atom has an electron pair, the recurring unit of Formula (I) above is cationic at low pH, and when R¹ is hydrogen, or an optionally substituted substituent selected from the group consisting of alkyl, alkenyl, alkynyl, heteroalkyl, heteroalkenyl, heteroalkynyl, aryl, heteroaryl, and heterocyclyl, the nitrogen atom has an associated positive charge.

In an embodiment, the recurring backbone degradable unit can have the following structure:

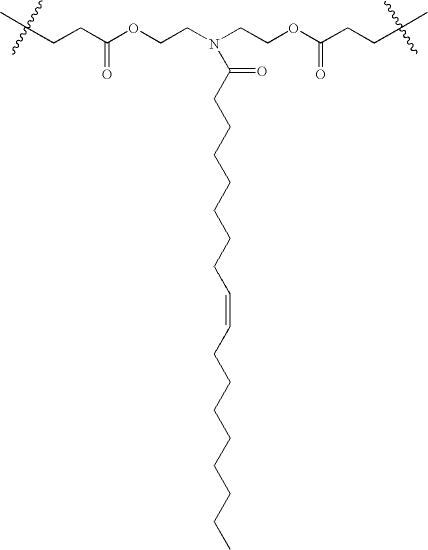

In preferred embodiments, the water soluble degradable crosslinked cationic polymer includes about 1 mole % to about 95 mole % of the recurring backbone degradable unit based on the total moles of recurring units in the water soluble degradable crosslinked cationic polymer. More preferably, the water soluble degradable crosslinked cationic polymer includes about 30 mole % to about 90 mole % of the recurring backbone degradable unit based on the total moles of recurring units in the water soluble degradable crosslinked cationic polymer. Yet more preferably, the water soluble degradable crosslinked cationic polymer includes about 50 mole % to about 86 mole % of the recurring backbone degradable unit based on the total moles of recurring units in the water soluble degradable crosslinked cationic polymer.

In preferred embodiments, the water soluble degradable crosslinked cationic polymer includes about 1 mole % to about 35 mole % of the recurring backbone cationic polyethyleneimine unit based on the total moles of recurring units in the water soluble degradable crosslinked cationic polymer. More preferably, the water soluble degradable crosslinked cationic polymer includes about 1 mole % to about 20 mole % of the recurring backbone cationic polyethyleneimine unit based on the total moles of recurring units in the water soluble degradable crosslinked cationic polymer. Yet more preferably, the water soluble degradable crosslinked cationic polymer includes about 5 mole % to about 15 mole % of the recurring backbone cationic polyethyleneimine unit based on the total moles of recurring units in the water soluble degradable crosslinked cationic polymer.

In preferred embodiments, the water soluble degradable crosslinked cationic polymer includes about 1 mole % to about 80 mole % of the recurring backbone polyethylene glycol unit based on the total moles of recurring units in the water soluble degradable crosslinked cationic polymer. Yet more preferably, the water soluble degradable crosslinked cationic polymer includes about 1 mole % to about 50 mole % of the recurring backbone polyethylene glycol unit based on the total moles of recurring units in the water soluble degradable crosslinked cationic polymer. Yet more preferably, the water soluble degradable crosslinked cationic polymer includes about 5 mole % to about 30 mole % of the recurring backbone polyethylene glycol unit based on the total moles of recurring units in the water soluble degradable crosslinked cationic polymer. Still more preferably, the water degradable crosslinked polymer includes about 8 mole % to about 30 mole % of the recurring backbone polyethylene glycol unit based on the total moles of recurring units in the water soluble degradable crosslinked cationic polymer.

An exemplary portion of a water soluble degradable crosslinked cationic polymer is shown below:

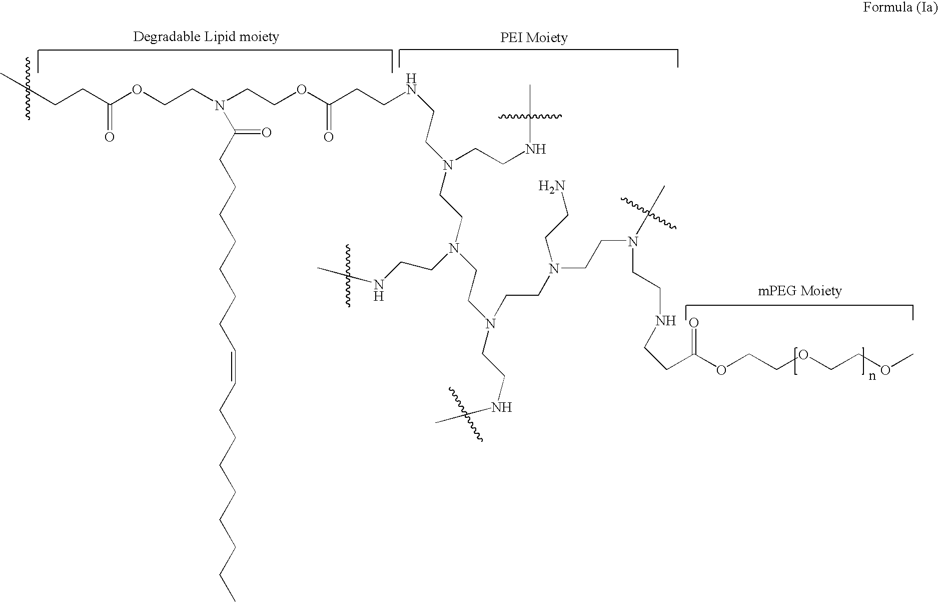

In an embodiment, a water soluble degradable crosslinked cationic polymer can include one or more branched PEI units in the backbone of the polymer having a molecular weight of about 1200 Daltons; one or more degradable units of Formula (I) in the backbone of the polymer; and one or more polyethylene glycol units in the backbone of the polymer having a molecular weight of about 454 Daltons.

A polymer which effectively delivers plasmid DNA into a cell cannot necessarily also effectively deliver siRNA into a cell. An uncorrelated factor of their delivery involves the difference in their molecular size: siRNA typically has around 21-23 base pairs (bp), whereas plasmid DNA has about 7,000-9,000 bp. See Kim et al. J. Control Release 2007 (in press). Carriers that may efficiently deliver a large circular macromolecule such as plasmid DNA may well be entirely unsuitable for short linear fragments such as siRNA.

Unless defined otherwise, all technical and scientific terms used herein have the same meaning as is commonly understood by one of ordinary skill in the art. All patents, applications, published applications and other publications referenced herein are incorporated by reference in their entirety. In the event that there are a plurality of definitions for a term herein, those in this section prevail unless stated otherwise.

As used herein, “Cₘ to Cₙ” in which “m” and “n” are integers refers to the number of carbon atoms in an alkyl, alkenyl or alkynyl group or the number of carbon atoms in the ring of a cycloalkyl, cycloalkenyl, cycloalkynyl, aryl, heteroaryl or heteroalicyclyl group. That is, the alkyl, alkenyl, alkynyl, ring of the cycloalkyl, ring of the cycloalkenyl, ring of the cycloalkynyl, ring of the aryl, ring of the heteroaryl or ring of the heteroalicyclyl can contain from “m” to “n”, inclusive, carbon atoms. Thus, for example, a “C₁ to C₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—. If no “m” and “n” are designated with regard to an alkyl, alkenyl, alkynyl, cycloalkyl cycloalkenyl, cycloalkynyl, aryl, heteroaryl or heteroalicyclyl group, the broadest range described in these definitions is to be assumed.

As used herein, “alkyl” refers to a straight or branched hydrocarbon chain fully saturated (no double or triple bonds) hydrocarbon group. The alkyl group may have 1 to 50 carbon atoms (whenever it appears herein, a numerical range such as “1 to 50” refers to each integer in the given range; e.g., “1 to 50 carbon atoms” means that the alkyl group may consist of 1 carbon atom, 2 carbon atoms, 3 carbon atoms, etc., up to and including 50 carbon atoms, although the present definition also covers the occurrence of the term “alkyl” where no numerical range is designated). The alkyl group may also be a medium size alkyl having 1 to 30 carbon atoms. The alkyl group could also be a lower alkyl having 1 to 5 carbon atoms. The alkyl group of the compounds may be designated as “C₁-C₄ alkyl” or similar designations. By way of example only, “C₁-C₄ alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl and the like.

The alkyl group may be substituted or unsubstituted. When substituted, the substituent group(s) is(are) one or more group(s) individually and independently selected from alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, ester, mercapto, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, and amino, including mono- and di-substituted amino groups, and the protected derivatives thereof.

As used herein, “alkenyl” refers to an alkyl group that contains in the straight or branched hydrocarbon chain one or more double bonds. An alkenyl group may be unsubstituted or substituted. When substituted, the substituent(s) may be selected from the same groups disclosed above with regard to alkyl group substitution unless otherwise indicated.

As used herein, “alkynyl” refers to an alkyl group that contains in the straight or branched hydrocarbon chain one or more triple bonds. An alkynyl group may be unsubstituted or substituted. When substituted, the substituent(s) may be selected from the same groups disclosed above with regard to alkyl group substitution unless otherwise indicated.

A “heteroalkyl” as used herein refers to an alkyl group as described herein in which one or more of the carbons atoms in the backbone of alkyl group has been replaced by a heteroatom such as nitrogen, sulfur and/or oxygen.

A “heteroalkenyl” as used herein refers to an alkenyl group as described herein in which one or more of the carbons atoms in the backbone of alkenyl group has been replaced by a heteroatom, for example, nitrogen, sulfur and/or oxygen.

A “heteroalkynyl” as used herein refers to an alkynyl group as described herein in which one or more of the carbons atoms in the backbone of alkynyl group has been replaced by a heteroatom such as nitrogen, sulfur and/or oxygen.

As used herein, “aryl” refers to a carbocyclic (all carbon) monocyclic or multicyclic aromatic ring system that has a fully delocalized pi-electron system. Examples of aryl groups include, but are not limited to, benzene, naphthalene and azulene. The ring of the aryl group may have 5 to 50 carbon atoms. The aryl group may be substituted or unsubstituted. When substituted, hydrogen atoms are replaced by substituent group(s) that is(are) one or more group(s) independently selected from alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxy, alkoxy, aryloxy, acyl, ester, mercapto, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, and amino, including mono- and di-substituted amino groups, and the protected derivatives thereof, unless the substituent groups are otherwise indicated.

As used herein, “heteroaryl” refers to a monocyclic or multicyclic aromatic ring system (a ring system with fully delocalized pi-electron system) that contain(s) one or more heteroatoms, that is, an element other than carbon, including but not limited to, nitrogen, oxygen and sulfur. The ring of the heteroaryl group may have 5 to 50 atoms. The heteroaryl group may be substituted or unsubstituted. Examples of heteroaryl rings include, but are not limited to, furan, furazan, thiophene, benzothiophene, phthalazine, pyrrole, oxazole, benzoxazole, 1,2,3-oxadiazole, 1,2,4-oxadiazole, thiazole, 1,2,3-thiadiazole, 1,2,4-thiadiazole, benzothiazole, imidazole, benzimidazole, indole, indazole, pyrazole, benzopyrazole, isoxazole, benzoisoxazole, isothiazole, triazole, benzotriazole, thiadiazole, tetrazole, pyridine, pyridazine, pyrimidine, pyrazine, purine, pteridine, quinoline, isoquinoline, quinazoline, quinoxaline, cinnoline, and triazine. A heteroaryl group may be substituted or unsubstituted. When substituted, hydrogen atoms are replaced by substituent group(s) that is(are) one or more group(s) independently selected from alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxy, alkoxy, aryloxy, acyl, ester, mercapto, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, and amino, including mono- and di-substituted amino groups, and the protected derivatives thereof.

As used herein, “cycloalkyl” refers to a completely saturated (no double bonds) mono- or multi-cyclic hydrocarbon ring system. When composed of two or more rings, the rings may be joined together in a fused, bridged or spiro-connected fashion. Cycloalkyl groups may range from C₃ to C₁₀, in other embodiments it may range from C₃ to C₈. A cycloalkyl group may be unsubstituted or substituted. Typical cycloalkyl groups include, but are in no way limited to, cyclopropyl, cyclobutyl, cyclopentyl, cyclohexyl, and the like. If substituted, the substituent(s) may be an alkyl or selected from those substituents indicated above with respect to substitution of an alkyl group unless otherwise indicated.

As used herein, “cycloalkenyl” refers to a cycloalkyl group that contains one or more double bonds in the ring although, if there is more than one, the double bonds cannot form a fully delocalized pi-electron system in the ring (otherwise the group would be “aryl,” as defined herein). When composed of two or more rings, the rings may be connected together in a fused, bridged or spiro-connected fashion. A cycloalkenyl group of may be unsubstituted or substituted. When substituted, the substituent(s) may be an alkyl or selected from the substituents disclosed above with respect to alkyl group substitution unless otherwise indicated.

As used herein, “cycloalkynyl” refers to a cycloalkyl group that contains one or more triple bonds in the ring. When composed of two or more rings, the rings may be joined together in a fused, bridged or spiro-connected fashion. A cycloalkynyl group may be unsubstituted or substituted. When substituted, the substituent(s) may be an alkyl or selected from the substituents disclosed above with respect to alkyl group substitution unless otherwise indicated.

As used herein, “heteroalicyclic” or “heteroalicyclyl” refers to a stable 3-to 18 membered ring which consists of carbon atoms and from one to five heteroatoms selected from the group consisting of nitrogen, oxygen and sulfur. The “heteroalicyclic” or “heteroalicyclyl” may be monocyclic, bicyclic, tricyclic, or tetracyclic ring system, which may be joined together in a fused, bridged or spiro-connected fashion; and the nitrogen, carbon and sulfur atoms in the “heteroalicyclic” or “heteroalicyclyl” may be optionally oxidized; the nitrogen may be optionally quaternized; and the rings may also contain one or more double bonds provided that they do not form a fully delocalized pi-electron system throughout all the rings. Heteroalicyclyl groups may be unsubstituted or substituted. When substituted, the substituent(s) may be one or more groups independently selected from the group consisting of alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, ester, mercapto, alkylthio, arylthio, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, haloalkyl, haloalkoxy, trihalomethanesulfonyl, trihalomethanesulfonamido, and amino, including mono- and di-substituted amino groups, and the protected derivatives thereof. Examples of such “heteroalicyclic” or “heteroalicyclyl” include but are not limited to, azepinyl, acridinyl, carbazolyl, cinnolinyl, 1,3-dioxin, 1,3-dioxane, 1,4-dioxane, 1,2-dioxolanyl, 13-dioxolanyl, 1,4-dioxolanyl, 1,3-oxathiane, 1,4-oxathiin, 1,3-oxathiolane, 1,3-dithiole, 1,3-dithiolane, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, trioxane, hexahydro-1,3,5-triazine, imidazolinyl, imidazolidine, isoxazoline, isoxazolidine, oxazoline, oxazolidine, oxazolidinone, thiazoline, thiazolidine, morpholinyl, oxiranyl, piperidinyl N-Oxide, piperidinyl, piperazinyl, pyrrolidinyl, pyrrolidone, pyrrolidione, 4-piperidonyl, pyrazoline, pyrazolidinyl, 2-oxopyrrolidinyl, tetrahydropyran, 4H-pyran, tetrahydrothiopyran, thiamorpholinyl, thiamorpholinyl sulfoxide, thiamorpholinyl sulfone, and their benzo-fused analogs (e.g., benzimidazolidinone, tetrahydroquinoline, 3,4-methylenedioxyphenyl).

Whenever a group is described as being “optionally substituted” that group may be unsubstituted or substituted with one or more of the indicated substituents. Likewise, when a group is described as being “unsubstituted or substituted” if substituted, the substituent may be selected from one or more the indicated substituents.

Unless otherwise indicated, when a substituent is deemed to be “optionally substituted,” or “substituted” it is meant that the substitutent is a group that may be substituted with one or more group(s) individually and independently selected from alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxy, alkoxy, aryloxy, acyl, ester, mercapto, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, and amino, including mono- and di-substituted amino groups, and the protected derivatives thereof. The protecting groups that may form the protective derivatives of the above substituents are known to those of skill in the art and may be found in references such as Greene and Wuts, Protective Groups in Organic Synthesis, 3^{rd} Ed., John Wiley & Sons, New York, N.Y., 1999, which is hereby incorporated by reference in its entirety.

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure or be stereoisomeric mixtures. In addition it is understood that, in any compound having one or more double bond(s) generating geometrical isomers that can be defined as E or Z each double bond may independently be E or Z a mixture thereof. Likewise, all tautomeric forms are also intended to be included.

The term “lipid” as used herein refers to fats and fatlike compounds. Exemplary lipids include fatty acids and sterols. A fatty acid is a long-chain monocarboxylic acid. A fatty acid can be saturated or unsaturated. A lipid is characterized as being essentially water insoluble, having a solubility in water of less than about 0.01% (weight basis). As used herein, the term “lipid group” refers to a lipid or portion thereof that has become directly attached to another group. For example, a lipid group may become attached to another compound (e.g., a monomer) by a chemical reaction between a functional group (such as a carboxylic acid group) on a fatty acid and an appropriate functional group on the monomer.

The term “crosslinked” as used herein refers to polymer chains that have been laterally linked together by bonds such as covalent bonds. As used herein, the term “crosslinked” is meant to encompass various degrees of crosslinking such as slightly crosslinked, moderately crosslinked and highly crosslinked.

Embodiments described herein relate to synthesis of the water soluble degradable crosslinked cationic polymers described herein. Lynn, et al. have described a method of synthesizing biodegradable cationic polymers using diacrylates as linker molecules between cationic compounds (see Lynn, et al. J. Am. Chem. Soc. 2001, 123, 8155-8156), which is hereby incorporated by reference in its entirety. In some embodiments, a water soluble degradable crosslinked cationic polymer can be synthesized by dissolving a first reactant comprising recurring ethyleneimine units in an organic solvent to form a dissolved or partially dissolved polymeric reactant; reacting the dissolved or partially dissolved polymeric reactant with a degradable monomeric reactant to form a degradable crosslinked polymer, wherein the degradable monomeric reactant comprises a lipid group; and reacting the degradable crosslinked polymer with a third reactant, wherein the third reactant comprises recurring polyethylene glycol units. For example, a water soluble degradable crosslinked cationic polymer that includes the recurring backbone degradable unit of Formula (I) can be synthesized by one method shown below. As shown in Scheme A, the compound of Formula (II) may be reacted PEI with to form a degradable crosslinked cationic polymer that includes one or moieties of Formula (III).

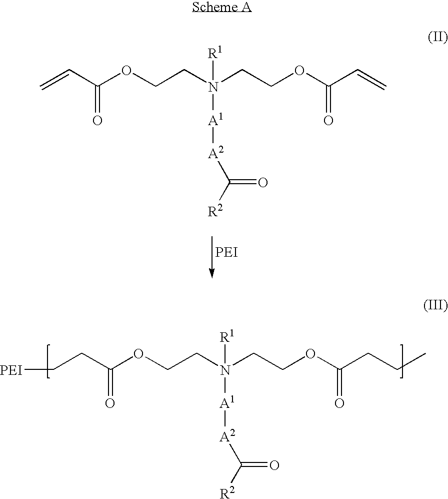

In Scheme A¹, A², R¹ and R² have the same meanings as described herein with respect to Formula (I).

The reaction illustrated in Scheme A may be carried out by intermixing the PEI and the compound of Formula (II) in a mutual solvent such as ethanol, methanol or dichloromethane with stirring; preferably at room temperature for several hours. The resulting polymer can be recovered using techniques known to those skilled in the art. For example, the solvent can be evaporated to recover the resulting polymer. This invention is not bound by theory, but it is believed that the reaction between the PEI and compound of Formula (II) involves a Michael reaction between one or more amines of the PEI with double bond(s) of the compound of Formula (II) (see J. March, Advanced Organic Chemistry 3^{rd} Ed., pp. 711-712 (1985)). The compound of Formula (II) shown in Scheme A may be prepared in the manner as described in U.S. Publication No. 2006/0258751, which is incorporated herein by reference, including all drawings.

The PEI can be linear or branched. The recurring backbone PEI units can have the structures of Formula (IV), (V), (VI) (VII) and/or (VIII).

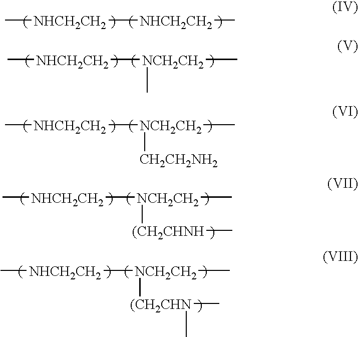

Various molecular weight of PEI can be used. When branched, the molecular weight of the recurring backbone PEI unit is preferably in the range of about 200 to 25,000 Daltons, more preferably 400 to 5,000 Daltons, yet more preferably in the range of about 600 to 2000 Daltons. When linear, the molecular weight of the recurring backbone PEI unit is preferably in the range of about 200 to 25,000 Daltons. In an embodiment, the linear recurring backbone PEI unit can have a molecular weight in the range of about 400 to about 1200 Daltons.

A variety of mole ratios of the degradable unit to PEI can be used to make the water soluble degradable crosslinked cationic polymer. In some embodiments, the mole ratio of the degradable monomeric reactant (e.g., a compound of Formula (II)) to PEI can be in the range of about 0.1:1 to about 50:1. In an embodiment, the mole ratio of the degradable monomeric reactant to PEI can be in the range of about 1:1 to about 30:1. In some embodiments, the mole ratio of the degradable monomeric reactant to PEI can be in the range of about 5:1 to about 25:1.

The moiety of Formula (III) can then be reacted with PEG or a derivative thereof such as mPEG (methoxypoly(ethylene glycol)), to form the water soluble degradable crosslinked cationic polymer. In some embodiments, the reaction is carried out at room temperature. The reaction products may be isolated by any means known in the art including chromatographic techniques. In an embodiment, the reaction product may be removed by precipitation followed by centrifugation.

Various molecular weights of PEG and derivatives thereof can be used. In some embodiments, the recurring backbone polyethylene glycol unit can have a molecular weight of about 50 Daltons to about 5,000 Daltons. In an embodiment, the recurring backbone polyethylene glycol unit can have a molecular weight of about 400 Daltons to about 600 Daltons.

The mole ratio of PEG to PEI can also vary. In some embodiments, the mole ratio of PEG to PEI can be in the range of about 0.1:1 to about 12:1. In some embodiments, the mole ratio of PEG to PEI can be in the range of about 1:1 to about 10:1. In some embodiments, the mole ratio of PEG to PEI can be in the range of about 1:1 to about 4:1.

When R¹ is hydrogen, or an optionally substituted substituent selected from the group consisting of alkyl, alkenyl, alkynyl, heteroalkyl, heteroalkenyl, heteroalkynyl, aryl, heteroaryl, and heterocyclyl, the compound of Formula (II) can be prepared by methods known to those skilled in the art. One method is shown below in Scheme B.

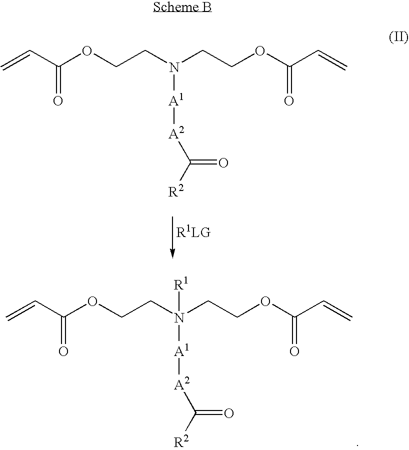

In Scheme B, A¹, A², R¹ and R² are the same as described herein, and LG is a suitable leaving group such as a halogen.

The weight average molecular weight of the water soluble degradable crosslinked cationic polymer can vary. In some embodiments, the weight average molecular weight may be in the range of about 500 Daltons to about 1,000,000 Daltons. In an embodiment, the weight average molecular weight may be in the range of about 2,000 Daltons to about 200,000 Daltons. The molecular weights may be determined by methods known to those skilled in the art, for example, by size exclusion chromatography using PEG standards or by agarose gel electrophoresis.

A wide variety of water soluble degradable crosslinked cationic polymers comprising the recurring backbone units described herein (e.g., Formula (I), PEI and PEG) may be made by varying the molecular weight and structure of the PEI, and the molecular weight and structure of the PEG, the size and type of the R¹ and R² groups on the compound of Formula (II), the A¹ and/or A² groups, and/or the mole ratios of the compound of Formula (II) to PEI and PEG. In addition, mixtures of different diacrylates and derivatives thereof and/or mixtures of different PEI's and/or mixtures of different PEG's may be used. In an embodiment, the methods described herein with respect to the synthesis of water soluble degradable crosslinked cationic polymers can be used to synthesize a polymer that includes portions of Formula (Ia) shown herein.

The water soluble degradable crosslinked cationic polymer is preferably biodegradable. A non-limiting list of degradable mechanisms include, but are not limited to, hydrolysis, enzyme cleavage, reduction, photo-cleavage, and/or sonication. This invention is not limited by theory, but it is believed that degradation of the degradable units of Formula (I) within the cell proceeds by enzymatic cleavage and/or hydrolysis of the ester linkages.

Embodiments described herein relate to a method of using the water soluble degradable crosslinked cationic polymer described herein to deliver RNA into cells. Preferably, the RNA is short interfering RNA (siRNA). RNA, and more specifically, siRNA, include RNA having 5 to 50 base pairs, preferably, 10 to 35 base pairs and more preferably 19 to 27 base pairs. RNA may also include mixed RNA/DNA molecules or mixed protein/RNA molecules. Delivery of the nucleic acid may be carried out in an aqueous solution or on a solid support.

Preferred embodiments are directed to transfection devices and methods which are simple, convenient and efficient compared to conventional transfection assays. A transfection device is made according to methods described herein by affixing a transfection reagent, such as a water soluble degradable crosslinked cationic polymer, on the solid surface of a cell culture device. In this preferred embodiment, there is no need to pre-mix the nucleic acid with a transfection reagent. This removes a key time-consuming step, which is required by conventional transfection procedures. Scientists only require approximately 40 minutes to complete the entire transfection process for 10 samples, compared to 2 to 5 hours or more required by conventional methods. This is particularly favorable for high throughput transfection assays, in which hundreds of samples will be tested at a time.

In preferred embodiments, transfection agents for coating a transfection device as described herein include but are not limited to water soluble degradable crosslinked cationic polymers, cationic polymers, lipopolymers, cationic pegylated polymers, pegylated lipopolymers, cationic lipids and pegylated cationic lipids. Examples of cationic polymer include but are not limited to CytoPure™ (Qbiogene), poly(lysine) and poly(arginine). Examples of lipopolymer reagents include but are not limited to jetPEI™ (Qbiogene). Examples of pegylated cationic polymers include but are not limited to PEI-PEG copolymers (Zhong, et al. (2005) Biomacromolecules vol. 6: 3440-3448, incorporated herein by reference), PEG-grafted cationic polymers (see U.S. Pat. No. 6,586,254, incorporated herein by reference) and the water soluble degradable crosslinked cationic polymer described herein. Examples of cationic lipid reagents include but are not limited to DOTAP (1,2-dioleoyl-3-(trimethyammonium) propane), Lipofectamine™ (Invitrogen), and siPORT™ (Ambion). Examples of pegylated cationic lipids include but are not limited to PEG-lipid complexes (Martin-Herranz, et al. (February 2004) Biophysical Journal vol. 86: 1160-1168, incorporated herein by reference). Additional cationic polymers useful in coating transfection devices are described in Table 1 below. In some embodiments, the transfection agent is a cationic pegylated polymer. In an embodiment, the cationic peglyated polymer can be a water soluble degradable crosslinked cationic polymer such as those described herein.

TABLE 1
Structures of cationic compounds and oligomers according to preferred embodiments

| Symbol | Name | Structure |
|---|---|---|
| C1 | Pentaethylenehexamine | 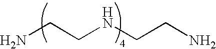 |
| C2 | Linear polyethylenimine(Mw = 423) | 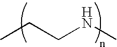 |
| C3 C4 | Branchedpolyethylenimine(Mw = 600)Branchedpolyethylenimine(Mw = 1200) | 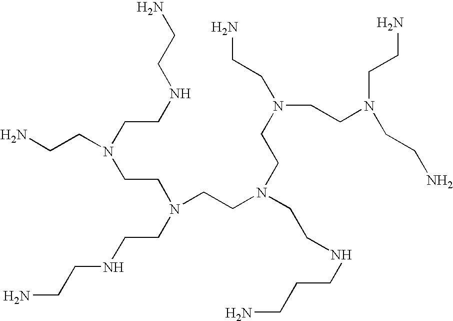 |
| C5 | N,N′-Bis(2-aminopropyl)-ethylenediamine | 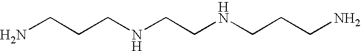 |
| C6 | Spermine | 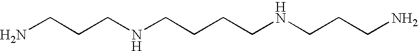 |
| C7 | N-(2-aminoethyl)-1,3-propanediamine | 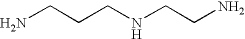 |
| C8 | N-(3-aminopropyl)-1,3-propanediamine | 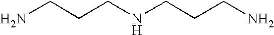 |
| C9 | N,N′-Bis(2-aminoethyl)-1,3-propanediamine | 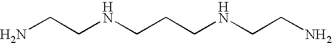 |
| C10 | Poly(amidoamine) | PAMA |
| | Dendrimer | |
| C11 | Poly(propyleneimine) | DAB-Am-16 |
| | dendrimer | |
| C12 | Spermidine | 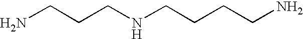 |
| C13 | 1,4-Bis(3-aminopropyl)piperazine | 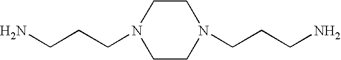 |
| C14 | 1-(2-Aminoethyl)piperazine | 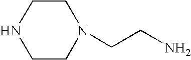 |
| C15 | Tri(2-aminoethyl)amine | 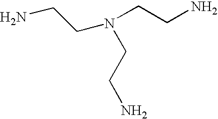 |
| C16 | Poly(L-lysine) | |

Preferred embodiments are directed to coating of the cationic pegylated polymer transfection agent, for example the water soluble degradable crosslinked cationic polymers described herein, onto a transfection device that is very easy to store, and which provides a simple method for siRNA delivery in which no siRNA/transfection reagent mixing step is required. The transfection procedure described herein can be finished in a short period of time, for instance approximately 40 minutes, and it provides a high throughput method for transfection in which large numbers of samples may be transfected at a time.

Embodiments of the method and device for gene suppression which are described herein overcome the common problems encountered in conventional transfection assays described above. The cationic pegylated polymer transfection reagents may be simply coated onto the surface of a cell culture device, which can be easily commercialized and mass-produced. Customers, researchers for instance, need only add a nucleic acid, such as siRNA of interest, directly to the surface of a cell culture device prior to transfection. Cells are then seeded on the surface of the cell culture device and incubated, without changing the medium, and the cells are analyzed. Changing medium during the transfection procedure is unnecessary. The methods described herein dramatically reduce the risk of error, by reducing the number of steps involved, thus increasing consistency and accuracy of the system.

In preferred embodiments, the transfection reagent is affixed on the surface of a slide or multi-well plate. However, a solid or semi-solid support of any shape may be used including but not limited to plates, filters and column packing material such as beads, fibers, and pellets of any shape and size.

Any suitable surface that can be used to affix the siRNA-containing mixture to its surface can be used. In some embodiments, semi-solid supports such as membranes (such as nitrocellulose, methylcellulose, PTFE or cellulose), and nylon filters and paper supports may be used.

The solid or semi-solid material for the support may be metal, non-metal, polymer or plastic, elastomer, or biologically derived material. Preferably the metal is gold, stainless steel, aluminum, nitinol, cobalt chrome, or titanium. Preferred non-metal materials include but are not limited to glass, silicon, silica, or ceramic.

Preferred plastic polymer and elastomer materials include but are not limited to polystyrene, polyacetal, polyurethane, polyester, polytetrafluoroethylene, polyethylene, polymethylmethacrylate, polyhydroxyethyl methacrylate, polyvinyl alcohol, polypropylene, polymethylpentene, polyetherketone, polyphenylene oxide, polyvinyl chloride, polycarbonate, polysulfone, acrylonitrile-butadiene-styrene, polyetherimide, polyvinylidene fluoride, and copolymers and combinations thereof. The material may be selected from polysiloxane, fluorinated polysiloxane, ethylene-propylene rubber, fluoroelastomer and combinations thereof. The material may be selected from polylactic acid, polyglycolic acid, polycaprolactone, polyparadioxanone, polytrimethylene carbonate and their copolymers.

In some embodiments, biologically-derived material such as protein, gelatin, agar, collagen, elastin, chitin, coral, hyaluronic acid, bone and combinations thereof may be utilized.

In some embodiments the solid or semi-solid support may include tissues (such as skin, endothelial tissue, bone, cartilage), or minerals (such as hydroxylapatite, graphite).

According to preferred embodiments the surfaces may be slides (glass or poly-L-lysine coated slides) or wells of a multi-well plate. In some embodiments, the solid or semi-solid surface may be an implantable device such as a stent.

By using this device, it is only necessary to add siRNA to the surface and allow the transfection reagent to form a complex with the siRNA. This reaction occurs in approximately 30 minutes. The cells are then seeded on the surface and incubated under suitable conditions for introduction of the siRNA into the cells. These steps may be carried out manually, by automated systems, or by a combination in which some steps are performed manually and others are automated.

For slides, such as a glass slide coated with poly-L-lysine (e.g. Sigma, Inc.), the transfection reagents are fixed on the surface and dried, and then a nucleic acid of interest such as double stranded siRNA is introduced. The slide is incubated at room temperature for 30 minutes to form siRNA/transfection reagent complexes on the surface of the transfection device. The siRNA/transfection reagent complexes create a medium for use in high throughput microarrays, which can be used to study hundreds to thousands of nucleic acids at the same time. In an alternative embodiment, the transfection reagents or drug delivery reagents can be affixed on the surface of the transfection device in discrete, defined regions to form a microarray of transfection reagents or drug delivery reagents. In this embodiment, molecules, such as nucleic acids, which are to be introduced into cells, are spread on the surface of the transfection device along with a transfection or delivery reagent. This method can be used in screening transfection reagents or other delivery reagents from thousands of compounds. The results of such a screening method can be examined through computer analysis.

In another embodiment, one or more wells of a multi-well plate may be coated with the cationic pegylated polymer transfection agent. Plates commonly used in transfection and drug screening are 96-well and 384-well plates. The cationic pegylated polymer transfection agent can be evenly applied to the bottom of plate. Hundreds of biomolecules such as siRNA are then added into the well(s) by, for instance, a multichannel pipette or automated machine. Results of transfection are then determined by using a microplate reader. This is a very convenient method of analyzing the transfected cells, because microplate readers are commonly used in most biomedical laboratories. The multi-well plate coated with cationic pegylated polymer transfection agent can be widely used in most laboratories to study gene regulation, gene function, molecular therapy, and signal transduction, as well as drug screening. Also, if different kinds of cationic pegylated polymer transfection agents are coated on the different wells of multi-well plates, the plates can be used to screen many cationic pegylated polymer transfection agents relatively efficiently. Recently, 1,536 and 3,456 well plates have been developed, which may also be used according to the methods described herein.

The transfection reagent or delivery reagent are preferably cationic pegylated polymer transfection agents which can introduce biomolecules, such as nucleic acids, preferably siRNA, into cells. Preferred embodiments use degradable cationic pegylated polymers such as the water soluble degradable crosslinked cationic polymers described herein.

Under appropriate conditions, the siRNA is added into the transfection device, which is coated with transfection or delivery reagent(s) such as a water soluble degradable crosslinked cationic polymer, to form biomolecule/delivery reagent complexes. The biomolecules are preferably dissolved in cell culture medium without fetal bovine serum and antibiotics, for example Dulbecco's Modified Eagles Medium (DMEM). If the transfection or delivery reagent is evenly affixed on the slide, the biomolecules can be spotted onto discrete locations on the slide. Alternatively, transfection or delivery reagents may be spotted on discrete locations on the slide, and the siRNA can simply be added to cover the whole surface of the transfection device. If the transfection reagent or delivery reagent are affixed on the bottom of multi-well plates, the siRNA is simply added into different wells by multi-channel pipette, automated device, or other method. The resulting product (transfection device coated with transfection or delivery reagent and siRNA) is incubated for 5 minutes to 3 hours, preferably 10 to 90 minutes, more preferably, 20-30 minutes at room temperature to form the siRNA/transfection reagent (or delivery reagent) complexes. In some cases, for example, different kinds of biomolecules are spotted on discrete location of the slide, the siRNA solution is removed to produce a surface bearing siRNA in complex with transfection reagent. In other cases, the siRNA solution is kept on the surface. Subsequently, cells in an appropriate medium and appropriate density are plated onto the surface. The resulting product (a surface bearing siRNA and plated cells) is maintained under conditions that result in entry of the biomolecules into plated cells.

Suitable cells for use according to the methods described herein include prokaryotes, yeast, or higher eukaryotic cells, including plant and animal cells, especially mammalian cells. In some embodiments, the cells are cancer cells. In some preferred embodiments, cell lines which are model systems for cancer are used, including but not limited to breast cancer (MCF-7, MDA-MB-438 cell lines), U87 glioblastoma cell line, B16F0 cells (melanoma), HeLa cells (cervical cancer), A549 cells (lung cancer) and rat tumor cell lines GH3 and 9L. In a most preferred embodiment B16F0 cells (melanoma) or HeLa cells (cervical cancer) are used as the test system. In preferred embodiments, the siRNA delivery agents are used to test the effectiveness of siRNAs on cancer as a treatment method.

Eukaryotic cells, such as mammalian cells (e.g., human, monkey, canine, feline, bovine, or murine cells), bacterial, insect or plant cells, are plated onto the transfection device, which is coated with transfection or delivery reagent and biomolecules, in sufficient density and under appropriate conditions for introduction/entry of the biomolecule into the eukaryotic cells and interaction of the biomolecule with cellular components. In particular embodiments the cells maybe selected from hematopoietic cells, neuronal cells, pancreatic cells, hepatic cells, chondrocytes, osteocytes, or myocytes. The cells are fully differentiated cells or progenitor/stem cells. The cells may be dividing cells, non-dividing cells, transformed cells, primary cells, or somatic cells.

In preferred embodiments, eukaryotic cells are grown in Dulbecco's Modified Eagles Medium (DMEM) containing 10% heat-inactivated fetal bovine serum (FBS) with L-glutamine and penicillin/streptomycin (pen/strep). It will be appreciated by those of skill in the art that certain cells should be cultured in a special medium, because some cells need special nutrition, such as growth factors and amino acids. The optimal density of cells depends on the cell types and the purpose of experiment. For example, a population of 70-80% confluent cells is preferred for gene transfection, but for oligonucleotide delivery the optimal condition is 30-50% confluent cells. In an example embodiment, if 5×10⁴ 293 cells/well were seeded onto a 96 well plate, the cells would reach 90% confluency at 18-24 hours after cell seeding. For HeLa 705 cells, only 1×10⁴ cells/well are needed to reach a similar confluent percentage in a 96 well plate.

After the cells are seeded on the surface containing siRNA/delivery reagent, the cells are incubated under optimal conditions for the cell type (e.g. 37° C., 5-10% CO₂). The culture time is dependent on the purpose of experiment. Typically, the cells are incubated for 24 to 48 hours for cells to express the target gene for gene transfection experiments. In the analysis of intracellular trafficking of siRNA in cells, minutes to several hours of incubation may be required and the cells can be observed at defined time points.

The results of siRNA delivery can be analyzed by different methods. In the case of gene transfection and antisense nucleic acid delivery, the target gene expression level can be detected by reporter genes, such as green fluorescent protein (GFP) gene, luciferase gene, or β-galactosidase gene expression. For example, the signal of GFP can be directly observed under a microscope, the activity of luciferase can be detected by a luminometer, and the blue product catalyzed by β-galactosidase can be observed under microscope or determined by a microplate reader. The practice of the invention is not limited to these examples. One of skill in the art is familiar with how these reporters function and how they may be introduced into a gene delivery system. The nucleic acid and its product, the protein, peptide, or other biomolecules delivered according to methods described herein and the target modulated by these biomolecules can be determined by various methods, such as detecting immunofluorescence or enzyme immunocytochemistry, autoradiography, or in situ hybridization. If immunofluorescence is used to detect expression of an encoded protein, a fluorescently labeled antibody that binds the target protein is used (e.g., added to the slide under conditions suitable for binding of the antibody to the protein). Cells containing the protein are then identified by detecting a fluorescent signal. If the delivered molecules can modulate gene expression, the target gene expression level can also be determined by methods such as autoradiography, in situ hybridization, and in situ PCR. However, the identification method depends on the properties of the delivered biomolecules, their expression product, the target modulated by it, and/or the final product resulting from delivery of the biomolecules.

Delivery methods may include spreading the polymer onto a surface such as a dish, slide or multiwell plate. The cells and siRNA may then be added in any order and incubated for a period of time effective for delivery of the siRNA into the cells.

### Delivery Enhancers

In some embodiments, the compositions may include delivery enhancers. It is generally recognized that there are three barriers to transport of a RNAi or siRNA biomolecule into the cell. These are the cell membrane, endosome membrane, and the release of the biomolecule from the carrier.

In the case of both DNA and RNA, the nucleic acid-carrier complex must first pass through the cell membrane. When this is accomplished by endocytosis, the nucleic acid-carrier complex is then internalized. The carrier along with the nucleic acid-cargo is enveloped by the cell membrane by the formation of a pocket and the pocket is subsequently pinched off. The result is a cell endosome, which is a large membrane-bound structure enclosing the nucleic acid cargo, and the carrier. The nucleic acid-carrier complex must then escape from the endosome membrane into the cytoplasm, and avoid enzyme degradation in the cytoplasm. The nucleic acid cargo must separate from the carrier. In general, anything designed to overcome one or more of the barriers described above may be considered a delivery enhancer.

In general, delivery enhancers fall into two categories. These are viral carrier systems and non-viral carrier systems. As human viruses have evolved ways to overcome the barriers to transport into the nucleus discussed above, viruses or viral components are useful in transport of nucleic acid into cells. One example of a viral component useful as a delivery enhancer is the hemagglutinin peptide (HA-peptide). This viral peptide facilitates transfer of biomolecules into cells by endosome disruption. At the acidic pH of the endosome, this protein causes release of the biomolecule and carrier into the cytosol.

Non-viral delivery enhancers may be either polymer-based or lipid-based. They are generally polycations which act to balance the negative charge of the nucleic acid. Branched chain versions of polycations such as PEI and Starburst dendrimers can mediate endosome release (Boussif, et al. (1995) Proc. Natl. Acad. Sci. USA vol. 92: 7297-7301). PEI is a highly branched polymer with terminal amines that are ionizable at pH 6.9 and internal amines that are ionizable at pH 3.9 and because of this organization, can generate a change in vesicle pH that leads to vesicle swelling and eventually, release from endosome entrapment.

Another means to enhance delivery is to design a ligand on the carrier. The ligand must have a receptor on the cell that has been targeted for cargo delivery. Biomolecule delivery into the cell is then initiated by receptor recognition. When the ligand binds to its specific cell receptor, endocytosis is stimulated. Examples of ligands which have been used with various cell types to enhance biomolecule transport are galactose, transferrin, the glycoprotein asialoorosomucoid, adenovirus fiber, malaria circumsporozite protein, epidermal growth factor, human papilloma virus capsid, fibroblast growth factor and folic acid. In the case of the folate receptor, the bound ligand is internalized through a process termed potocytosis, where the receptor binds the ligand, the surrounding membrane closes off from the cell surface, and the internalized material then passes through the vesicular membrane into the cytoplasm (Gottschalk, et al. (1994) Gene Ther 1:185-191).

Various agents have been used for endosome disruption. Besides the HA-protein described above, defective-virus particles have also been used as endosomolytic agents (Cotten, et al. (July 1992) Proc. Natl. Acad. Sci. USA vol. 89: pages 6094-6098). Non-viral agents are either amphiphillic or lipid-based.

The release of biomolecules such as DNA into the cytoplasm of the cell can be enhanced by agents that either mediate endosome disruption, decrease degradation, or bypass this process all together. Chloroquine, which raises the endosomal pH, has been used to decrease the degradation of endocytosed material by inhibiting lysosomal hydrolytic enzymes (Wagner, et al. (1990) Proc Natl Acad Sci USA vol. 87: 3410-3414). Branched chain polycations such as PEI and starburst dendrimers also promote endosome release as discussed above.

To completely bypass endosomal degradation, subunits of toxins such as Diptheria toxin and Pseudomonas exotoxin have been utilized as components of chimeric proteins that can be incorporated into a gene/gene carrier complex (Uherek, et al.(1998) J. Biol. Chem. vol. 273: 8835-8841). These components promote shuttling of the nucleic acid through the endosomal membrane and back through the endoplasmic reticulum.

### Methods of Use

One embodiment disclosed herein relates to a method of treating cancer comprising using the water soluble degradable crosslinked cationic polymers described herein to deliver siRNA into mammalian cancer cells for the treatment of cancer. Exemplary cancers include cervical cancer, melanoma, prostate cancer, lung cancer, colorectal cancer, leukemia, pancreatic cancer endometrial cancer, ovarian cancer or non-Hodgkin lymphoma.

In an embodiment, siRNA is administered as a disease treatment. In preferred embodiments, siRNA corresponding to all or part of a coding region of a gene that is expressed or overexpressed in a disease state is administered to a patient in need of treatment. In preferred embodiments, siRNA corresponding to all or part of a gene encoding a protein elevated in cardiovascular disease or diabetes is administered to a subject to bring levels of the gene product into or closer to a normal and/or health range. In a preferred embodiment, siRNA to Apolipoprotein-B (Apo-B) is administered in a complex with a transfection agent described herein, such as the water soluble degradable crosslinked cationic polymers described herein. Administration of siRNA corresponding to the coding region of Apolipoprotein-B (Apo-B) may lower risk of cardiovascular disease, myocardial infarction and/or stroke.

As used herein, a “subject” refers to an animal that is the object of treatment, observation or experiment. “Animal” includes cold- and warm-blooded vertebrates and invertebrates such as fish, shellfish, reptiles, and, in particular, mammals. “Mammal” includes, without limitation, mice, rats, rabbits, guinea pigs, dogs, cats, sheep, goats, cows, horses, primates, such as monkeys, chimpanzees, and apes, and, in particular, humans.

As used herein, the terms “treating,” “treatment,” “therapeutic,” or “therapy” do not necessarily mean total cure or abolition of the disease or condition. Any alleviation of any undesired signs or symptoms of a disease or condition, to any extent can be considered treatment and/or therapy. Furthermore, treatment may include acts that may worsen the patient's overall feeling of well-being or appearance.

The term “therapeutically effective amount” is used to indicate an amount of an active compound, or pharmaceutical agent, that elicits the biological or medicinal response indicated. This response may occur in a tissue, system, animal or human and includes alleviation of the symptoms of the disease being treated.

The exact formulation, route of administration and dosage for the composition and pharmaceutical compositions disclosed herein can be chosen by the individual physician in view of the patient's condition. (See e.g., Fingl et al. 1975, in “The Pharmacological Basis of Therapeutics”, Chapter 1, which is hereby incorporated by reference in its entirety). Typically, the dose range of the composition administered to the patient can be from about 0.5 to 1000 mg/kg of the patient's body weight, or 1 to 500 mg/kg, or 10 to 500 mg/kg, or 50 to 100 mg/kg of the patient's body weight. The dosage may be a single one or a series of two or more given in the course of one or more days, as is needed by the patient. Where no human dosage is established, a suitable human dosage can be inferred from ED₅₀ or ID₅₀ values, or other appropriate values derived from in vitro or in vivo studies, as qualified by toxicity studies and efficacy studies in animals.

Although the exact dosage will be determined on a drug-by-drug basis, in most cases, some generalizations regarding the dosage can be made. The daily dosage regimen for an adult human patient may be, for example, an oral dose of between 0.1 mg and 500 mg of each ingredient, preferably between 1 mg and 250 mg, e.g. 5 to 200 mg or an intravenous, subcutaneous, or intramuscular dose of each ingredient between 0.01 mg and 100 mg, preferably between 0.1 mg and 60 mg, e.g. 1 to 40 mg of each ingredient of the pharmaceutical compositions disclosed herein or a pharmaceutically acceptable salt thereof calculated as the free base, the composition being administered 1 to 4 times per day. Alternatively the compositions disclosed herein may be administered by continuous intravenous infusion, preferably at a dose of each ingredient up to 400 mg per day. Thus, the total daily dosage by oral administration of each ingredient will typically be in the range 1 to 2000 mg and the total daily dosage by parenteral administration will typically be in the range 0.1 to 400 mg. In some embodiments, the compounds will be administered for a period of continuous therapy, for example for a week or more, or for months or years.

Dosage amount and interval may be adjusted individually to provide plasma levels of the active moiety, which are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations.

Dosage intervals can also be determined using MEC value. Compositions should be administered using a regimen, which maintains plasma levels above the MEC for 10-90% of the time, preferably between 30-90% and most preferably between 50-90%.

The amount of composition administered will, of course, be dependent on the subject being treated, on the subject's weight, the severity of the affliction, the manner of administration and the judgment of the prescribing physician.

### EXAMPLES

All the chemicals, methanol, dichloromethane (DCM), polyethylene glycol methyl ether acrylate (PEG), and other reagents were purchased from Sigma-Aldrich chemical company. Polyethylenimine was purchased from PolyScience, Inc. The degradable monomeric reactant of Formula (II) was synthesized according to the general procedure reported in patent application U.S. application Ser. No. 11/216,986 (US Publication No. 2006/0258751) which is incorporated herein by reference.

HeLa human cervix adenocarcinoma and B16F0 mouse skin melanoma cells were purchased from ATCC and cultured in DMEM medium with 10% FBS. GFP-expression stable cell lines were generated by transfecting GFP expression vectors into the cells and selected by hygromycin B (for HeLa-GFP) or neomycin (for B16F0-GFP).

### Example 1

### Synthesis of Water Soluble Degradable Crosslinked Cationic Polymers

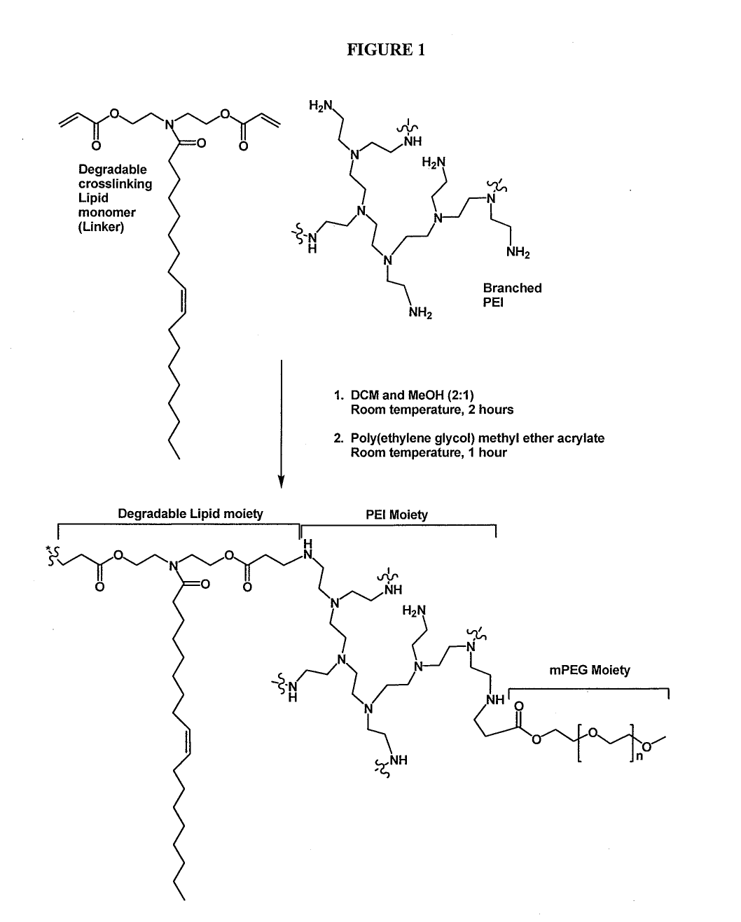

The schematic outline of synthesis is shown in FIG. 1. PEI (30 mg) was dissolved in methanol (3 mL). A solution of a degradable monomeric reactant of Formula (II) (36 mg) in DCM (dichloromethane) (6 mL) was added into the PEI solution. The mixture was stirred for 4 hours. A solution of mPEG (23 mg) in DCM (2 mL) was added to the mixture. After addition, the mixture was stirred for another 4 hours. The reaction mixture was quenched by adding 2 M hydrochloric acid in diethyl ether. A white precipitate was formed, isolated by centrifugation, and washed with diethyl ether. The water soluble degradable crosslinked cationic polymer product (65 mg, 74% yield) was obtained after drying with high vacuum. The product was confirmed with ¹H-NMR.

### Example 2

### Synthesis of Water Soluble Degradable Crosslinked Cationic Polymers

The schematic outline of synthesis is shown in FIG. 1. PEI (15 mg) was dissolved in methanol (3 mL). A solution of a degradable monomeric reactant of Formula (II) (71 mg) in DCM (6 mL) was added into the PEI solution. The mixture was stirred for 4 hours. A solution of mPEG (11 mg) in DCM (2 mL) was added to the mixture. After addition, the mixture was stirred for another 4 hours. The reaction mixture was quenched by adding 2 M hydrochloric acid in diethyl ether. A white precipitate was formed, isolated by centrifugation, and washed with diethyl ether. The water soluble degradable crosslinked cationic polymer product (65 mg, 74% yield) was obtained after drying with high vacuum. The product was confirmed with ¹H-NMR.

### Example 3

### Synthesis of Water Soluble Degradable Crosslinked Cationic Polymer: Polymer 1

A solution of branched PEI (MW=1200 Daltons, 0.960 g, 0.80 mmol) in a mixture of dichloromethane:methanol (1:2, 8 mL) was added to a solution of a degradable monomeric reactant of Formula (II) (1.91 g, 4.0 mmol) in dichloromethane:methanol (1:2, 40 mL). Before addition, the flask containing a degradable monomeric reactant of Formula (II) was washed with dichloromethane:methanol (1:2, 0.5 mL×4 times). After addition was complete, the reaction mixture was stirred at room temperature for 2 hours.

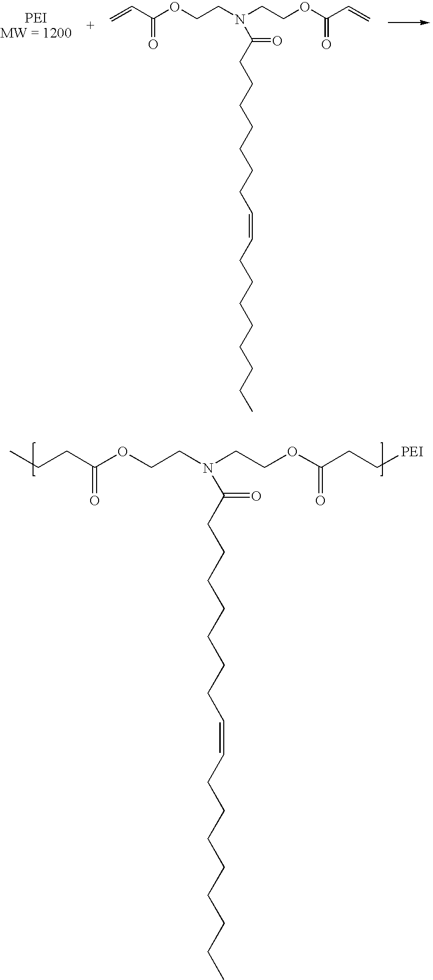

A solution of mPEG (MW=454 Daltons, 0.726 g, 1.6 mmol) in dichloromethane:methanol (1:2, 3 mL) was then added. Before addition, the flask containing the mPEG was washed with dichloromethane:methanol (1:2, 0.5 mL×4 times). The reaction mixture was then stirred for additional hour.

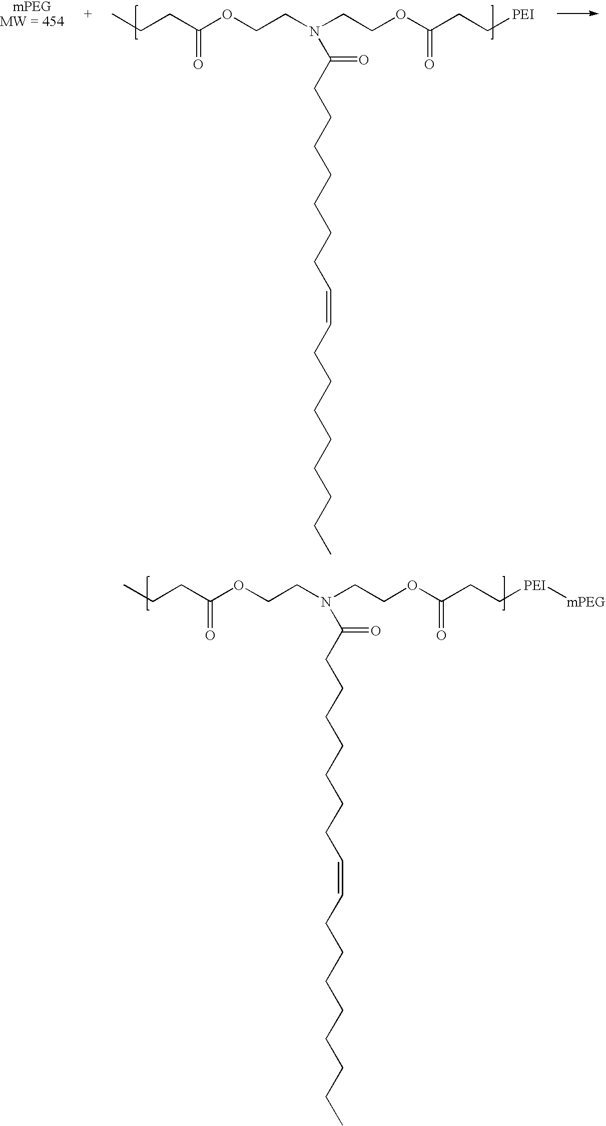

The reaction was then cooled in ice-water for 10 minutes before being quenched with a solution of 2 M hydrochloric acid in ether (30 mL) while stirring. The suspension was placed in eight 50-mL conical centrifuge tubes and diluted with additional cooled ether (−20° C.). The suspension in the tubes was centrifuged. The liquid was decanted, and the white solid product was washed with more ether and centrifuged twice. The product was dried under vacuum to yield 3.97 grams (90%). The product, polymer 1 (degradable lipid unit:PEI:PEG (5:1:2), was characterized by ¹H-NMR.

### Example 4

### siRNA Transfection

Cells expressing Green Fluorescent Protein (GFP) were seeded to 96-well plates at a density of 1×10⁴ cells per well one day before the transfection. A solution of siRNA (1.0 μg) was dissolved in distilled water and further diluted to 30 μL with OptiMEM (Invitrogen). The siRNA used in these experiments was anti-GFP (CGAGAAGCGCGAUCACAUGUU (SEQ ID NO: 1). Selected water soluble degradable crosslinked cationic polymers [polymer 2 (degradable unit:PEI:PEG (12:1:2)), polymer 3 (degradable unit:PEI:PEG (16:1:2)), polymer 4 (degradable unit:PEI:PEG (17:1:2)), polymer 5 (degradable unit:PEI:PEG (20:1:2)), and controls [PEI1200, Cytopure™, Lipofectamine 2000™, and degradable unit:PEI (5:1), all molar ratios] were prepared at a concentration of 5 mg/ml, by dissolving the delivery reagents in appropriate amount of dH₂O. During the experiment, according to the compound to siRNA ratio specified in the experiment, the delivery reagent solutions were further diluted with OptiMEM to a final volume of 30 μl. The diluted siRNA solution and the delivery reagent solutions were mixed and incubated at room temperature for 15 min. The mixture of the siRNA and the delivery reagents (15 μL) was added to each well of the pre-seeded cells, mixed, and incubated at 37° C. incubator with 5% CO₂. After 48 hours, transfection and efficiency cell viability were evaluated.

### Example 5

### Evaluation of Transfection Efficiency

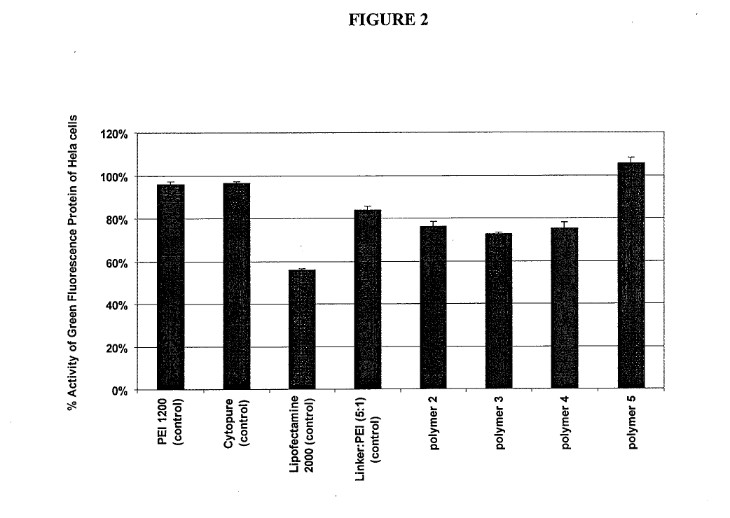

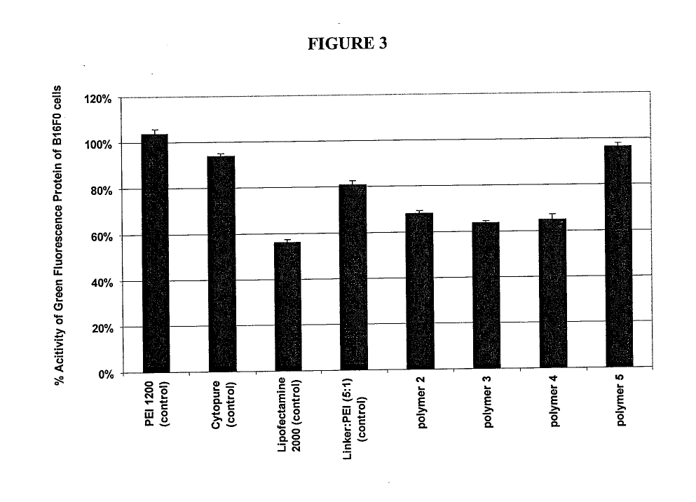

After about 48 hours, transfection was evaluated by measuring the expression of GFP under the fluorescence microscope. The absorbance of GFP was detected at 485-528 nm with the UV-vis microplate reader. The results of percent activity of green fluorescence protein in Hela cells are presented in FIG. 2. The results of percent activity of green fluorescence protein in B16F0 cells are presented in FIG. 3. The results show that water soluble degradable crosslinked cationic polymers inhibit (or silence) green fluorescence protein expression more effectively compared to the controls [PEI1200, Cytopure™, and crosslinked degradable unit:PEI (5:1)].

### Example 6

### Cell Viability Assay

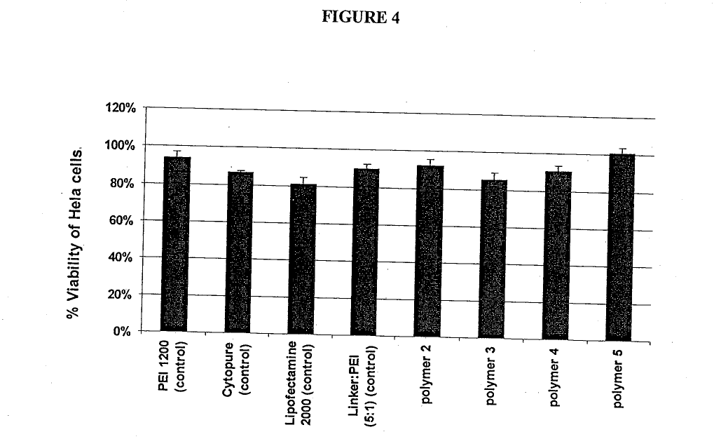

A solution 3-(4,5-dimethylthiazol-2-yl)-2,5-diphenyltetrazolium bromide (MTT) was prepared by dissolving 250 mg of solid MTT in 50 mL of Dubecco PBS and stored at 4° C. After 48 hours of transfection, MTT solution (10 μL of the 5 mg/mL) was added to each well of the cells and incubated at 37° C. for 2-4 hours until purple crystal growth could be observed. Then solubilized solution (100 μL) was added and incubated at 37° C. overnight. The absorbance was detected at wavelength of 570 nm with the absorbance at 690 nm as reference. The results of cell viability assay are presented in FIG. 4.

### Example 7

### Plate Coating

PEI-1.2K, Cytopure™, L2K, and polymer 1 were separately dissolved in H₂O to make 5 mg/mL stock solutions. Different compounds were coated onto 96-well plates at amounts of 0.625 μg, 1.25 μg, 2.50 μg and 5.0 μg per well in 30 μL final volume and dried in vacuum-drier overnight. The dried plates were sealed in aluminum foil until use.

### Example 8

### Transfection

1.0 μg siRNA (anti-GFP) was diluted to 30 μL with OptiMEM (Invitrogen) and was added to the coated wells and incubated at room temperature for 25 minutes. Cells (expressing GFP) were then seeded to the correspondent wells at 1.5×10⁴ per well in 100 μL culture medium and incubated at 37° C. incubator with 5% CO₂.

### Example 9

### Detection

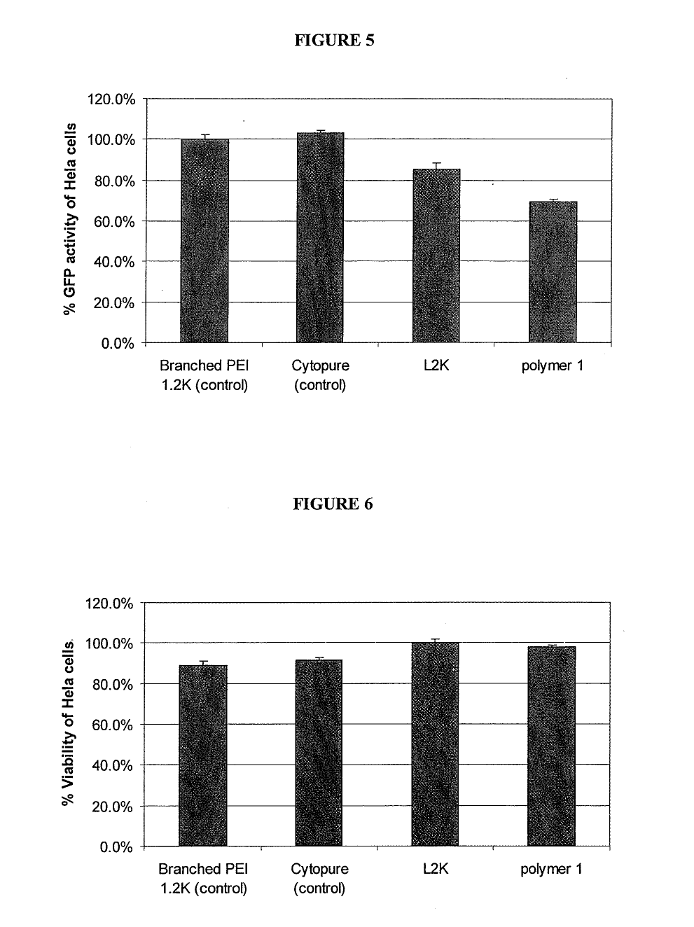

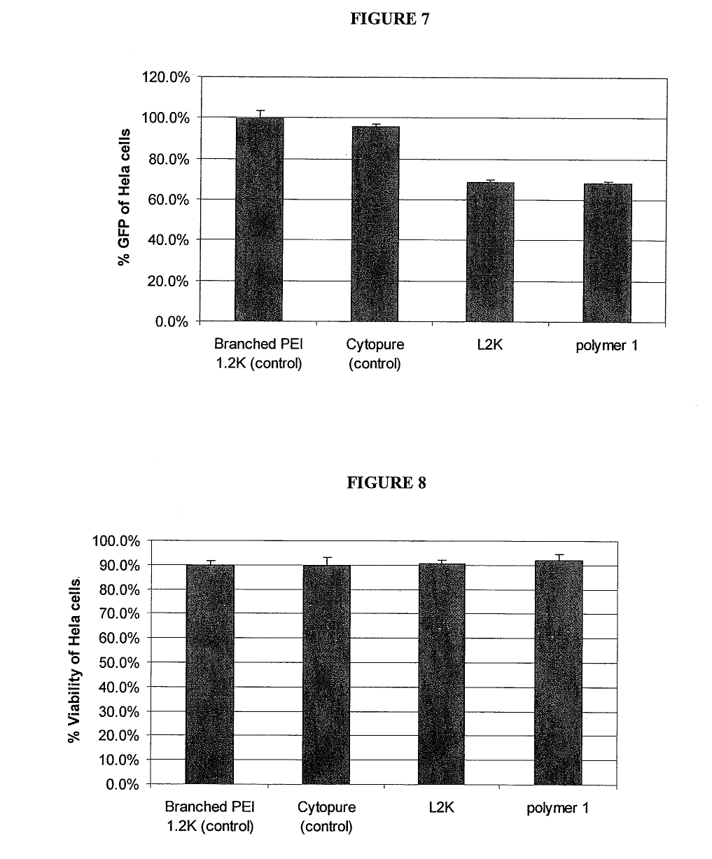

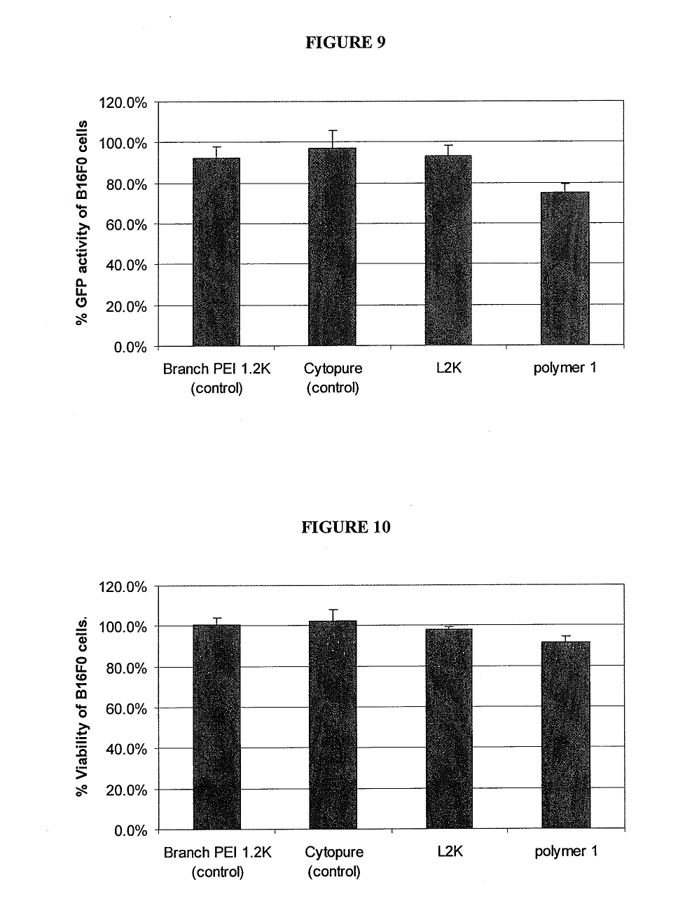

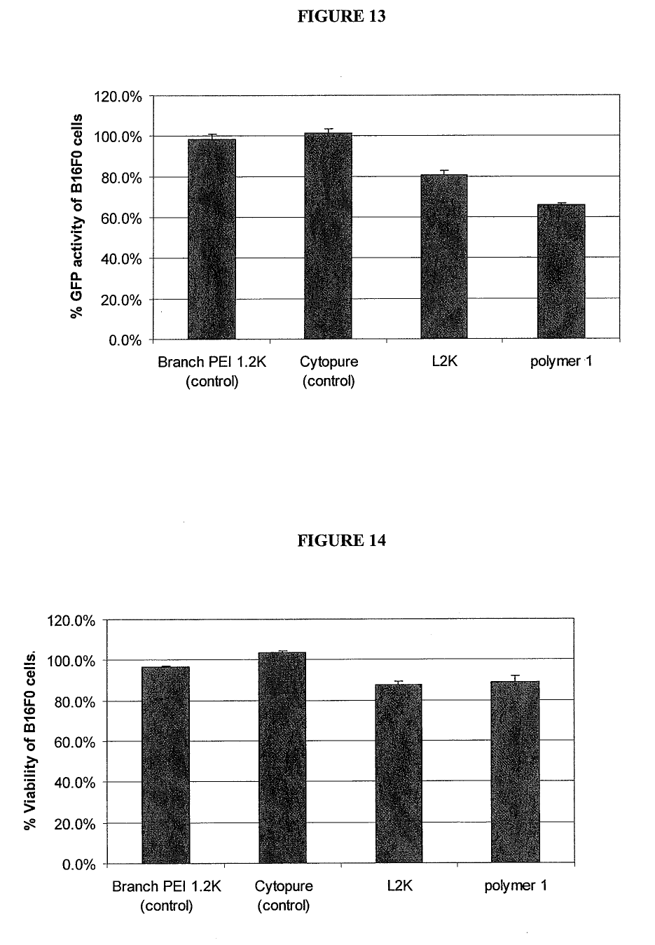

After 48 hours of the transfection, the expression of GFP was observed under the microscope. The absorbance of GFP was detected at 485-528 nm with the UV/vis microplate reader. The results are reported in FIGS. 5, 7, 9, 11, and 13 at ratios of polymer:siRNA of 5:1 and 10:1 for Hela cells and ratios of 2.5:1, 5:1 and 10:1 for B16F0 cells. In all cases, transfection using polymer 1 was better (Hela cells 5:1; B16F0, all ratios tested) or comparable (Hela cells, 10:1 ratio) compared to the positive control (Lipofectamine™).

### Example 10

### Cell Viability Assay

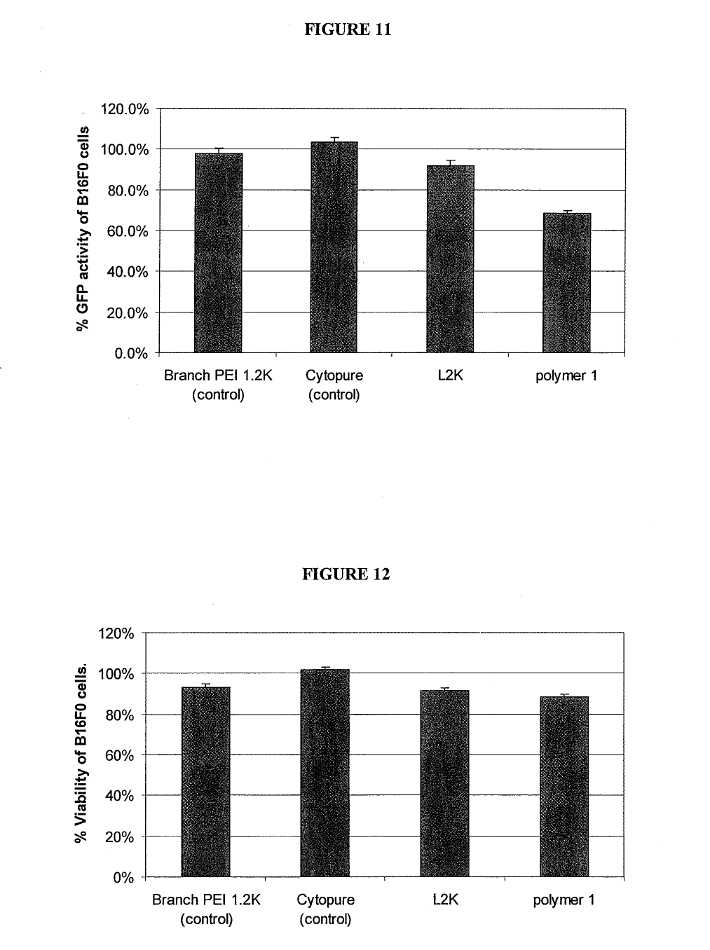

After 48 hours of the transfection, 10 μL of the 3-(4,5-Dimethylthiazol-2-yl)-2,5-diphenyltetrazolium bromide (MTT, 5 mg/mL in PBS) solution was added to each well of the cells, and the cells were incubated at 37° C. for 2-4 hours until purple crystal growth could be observed. 100 μL solubilization solution was then added into each well and each well was incubated at 37° C. overnight. The absorbance was detected at wavelength of 570 nm with the absorbance at 690 nm as reference. The results are reported in FIGS. 6, 8, 10, 12, and 14 at ratios of polymer:siRNA of 5:1 and 10:1 for Hela cells and ratios of 2.5:1, 5:1 and 10:1 for B16F0 cells. At all tested ratios for both cell types, polymer 1 did not display cytotoxicity.

### Example 11

### Inhibition of Apolipoprotein-B Protein by siRNA in HepG2 Cells

Apolipoprotein-B (Apo-B) is the primary apolipoprotein of low density lipoproteins and is a marker for heart disease risk. Inhibition of Apo-B expression may reduce risk of heart disease.

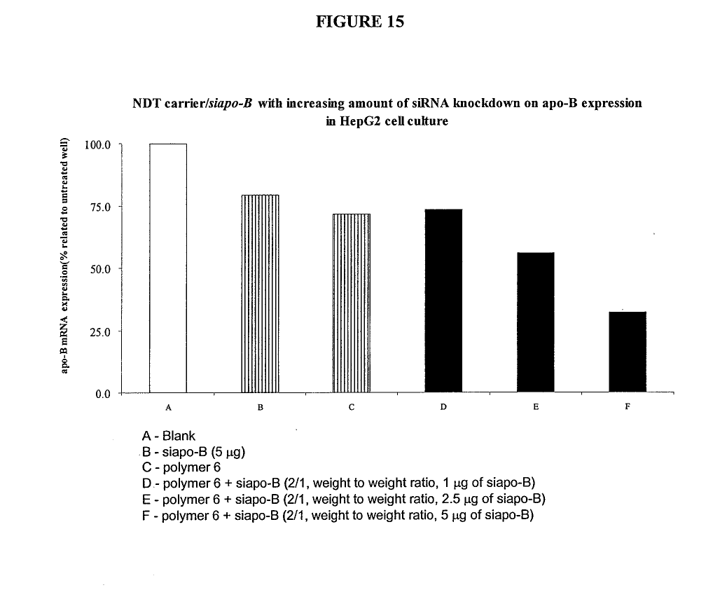

Polymer 6 was synthesized as described in Examples 1-3. Polymer 6 is a water soluble degradable crosslinked cationic polymer where the molar ratio of degradable unit:PEI:PEG is 16.5:1:2. The degradable unit and PEI are the same as described in Example 3. Polymer 6 was used as the transfection agent in the experiments of FIGS. 15 and 17-20.

1, 2.5 or 5 μg of siRNA (anti-Apo-B, synthesized at Dharmacon, with the sequences of sense: 5′-GUCAUCACACUGAAUACCAAUUU-3′ (SEQ ID NO: 2) and antisense: 5′-AUUGGUAUUCAGUGUGAUGACUU-3′) (SEQ ID NO: 3) (anti-Apo-B) was diluted to 30 μL with OptiMEM™ (Invitrogen) complexed with the pegylated polymer 6 as the transfection agent as described in Example 4 above. The ratio of transfection agent: siRNA was 2:1. The mixture was added to 96-well plates contained HepG2 cells that were seeded to the wells at 1.5×10⁴ per well in 100 μL culture media and incubated at 37° C. incubator with 5% CO₂. The control treatments were (1) no siRNA and no polymer (Blank), (2) anti-Apo-B only (siapoB alone: 5 μg), and (3) the transfection agent alone (polymer 6 alone).

After 48 hours incubation, mRNA expression was determined by Quantitative RT-PCR with the primer for Apo-B mRNA, forwarded as 5′-TTTGCCCTCAACCTACCAAC-3′ (SEQ ID NO: 4) and reversed as 5′-TGCGATCTTGTTGGCTACTG-3′ (SEQ ID NO: 5). FIG. 15 shows the effects of siRNA on expression of Apo-B mRNA in HepG2 cell culture. Expression is shown relative to the Blank. As expected neither siApo-B alone nor the transfection agent (polymer 6) alone had any effect on expression of Apo-B in HepG2 cells. As the amount of the transfection agent/siRNA complex increases, the inhibition of Apo-B mRNA levels in the HepG2 cells decreases showing that the cationic peglyated polymer transfection agent is effective in delivery of anti-Apo-B to mammalian cells to inhibit Apo-B in vitro.

### Example 12

### Stability of Transfection Agent siRNA Complexes in 5% Glucose

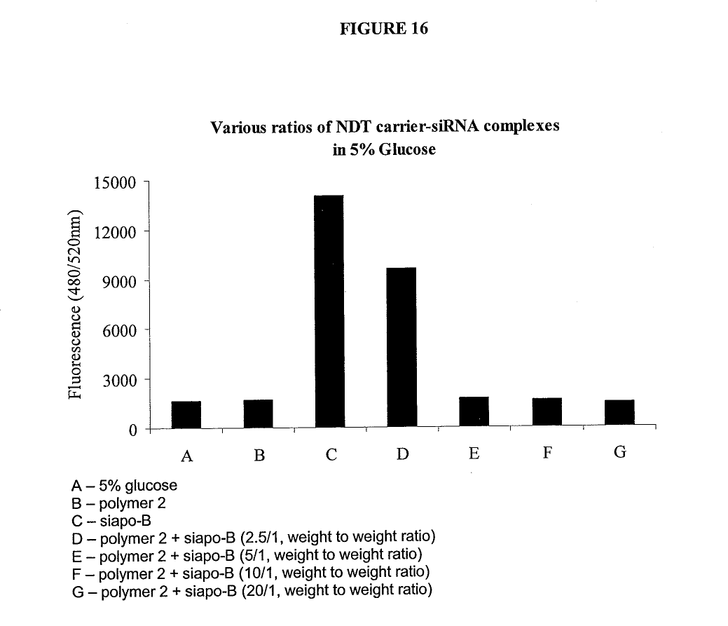

FIG. 16 shows the stability of the transfection agent/siRNA complexes as demonstrated by Fluorescence after binding to RiboGreen™ (Invitrogen). The results show that as the ratio of transfection agent (polymer 2) to siRNA increases, fluorescence decreases.

### Example 13

### Effect of Anti-siRNA in nu/nu Mice

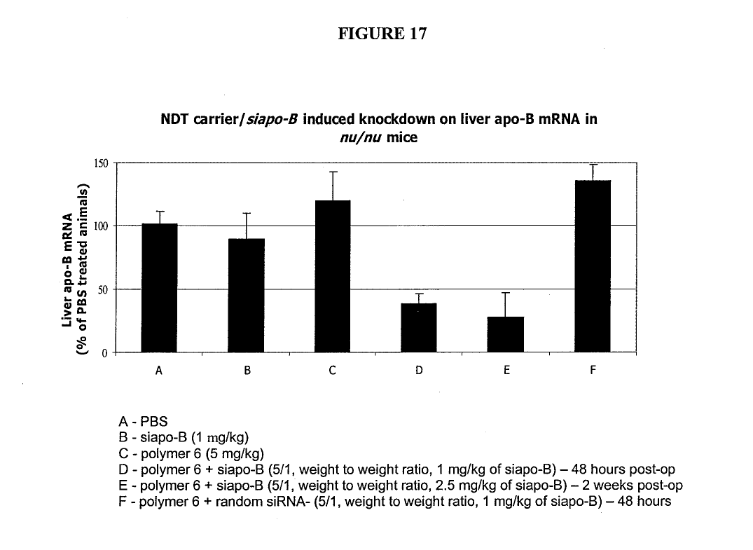

Complexes were formed between transfection agent polymer 6 and siRNA (anti-Apo-B) at a ratio of 5:1, prepared as described in Example 11 above. The complexes were injected subcutaneously into the tail vein of the mice. Results are shown in FIG. 17.

As seen in FIG. 17, both administration of 1.0 mg/kg anti-Apo-B siRNA at 48 hours post-op and 2.5 mg/kg anti-Apo-B siRNA at 2 weeks post-op effectively inhibited expression of Apo-B mRNA in nu/nu mice.

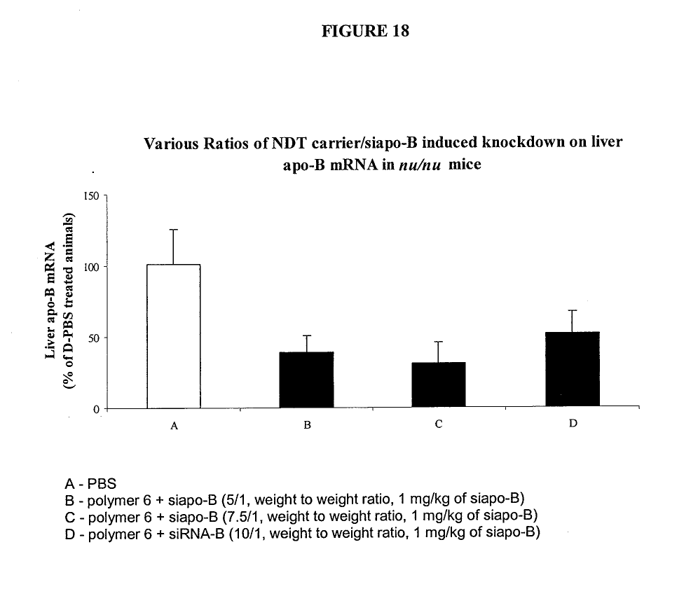

In a separate experiment, the ratio of transfection agent (polymer 6) to anti-Apo-B siRNA was varied from 5:1 to 10:1. The amount injected was maintained at 1.0 mg/kg. Although all ratios inhibited expression of Apo-B mRNA, the strongest inhibition was observed at ratios of 5:1 and 7.5:1 (FIG. 18).

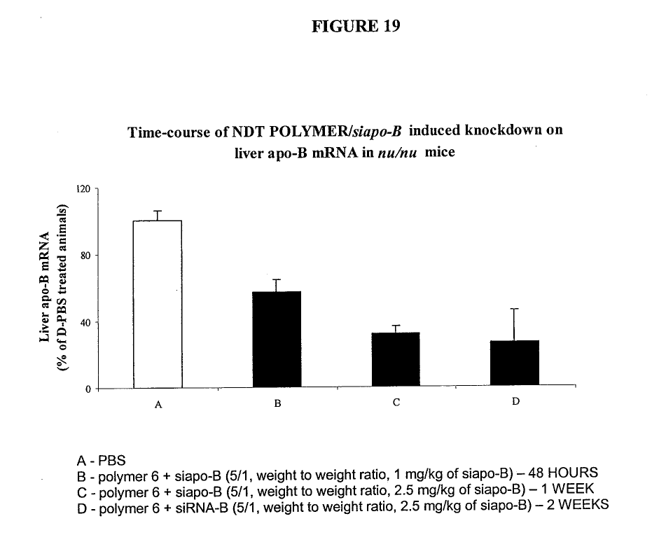

The inhibitory effect of the injection of the cationic peglyated polymer into the mice persisted for at least 2 weeks as shown in FIG. 19 with an injection amount of 2.5 mg/kg.

### Example 14

### Effect of Anti-siRNA in C57BL/6 Mice

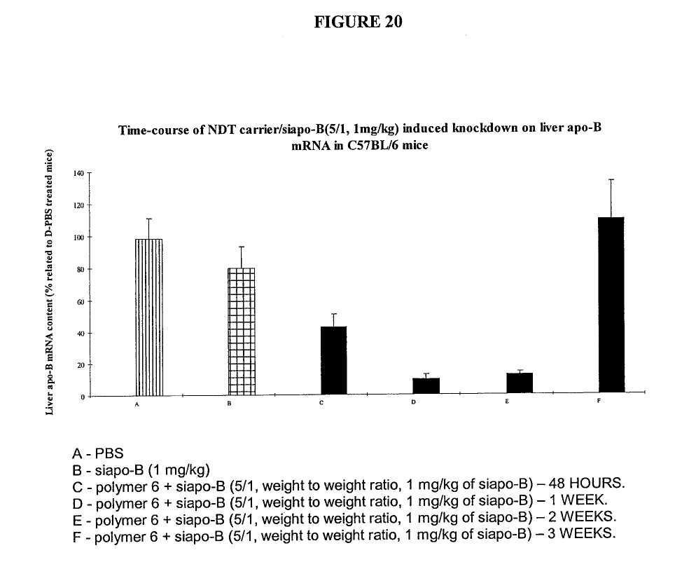

FIG. 20 shows injection of 1.0 mg/kg anti-Apo-B siRNA complexed with transfection agent polymer 6 into a general purpose mice strain (C57BL/6). When the amount injected was increased to 1.0 mg/kg relative to the nu/nu mice of Example 13, stronger inhibition of Apo-B mRNA expression was observed (FIG. 20). The inhibition was observed for 2 weeks. At three weeks, levels of Apo-B expression returned to control levels (FIG. 20).

It will be understood by those of skill in the art that numerous and various modifications can be made without departing from the spirit of the present invention. Therefore, it should be clearly understood that the forms of the present invention are illustrative only and are not intended to limit the scope of the present invention.

What is claimed is:

1. A composition for siRNA delivery comprising a water soluble degradable crosslinked cationic polymer comprising:
(a) a recurring backbone polyethylene glycol (PEG) unit,
(b) a recurring backbone cationic polyethyleneimine (PEI) unit, and
(c) a recurring backbone degradable unit that comprises a side chain lipid group.

2. The composition of claim 1, wherein the recurring backbone polyethylene glycol unit has a molecular weight in the range of about 50 to about 5,000 Daltons.

3. The composition of claim 1, wherein the recurring backbone cationic polyethyleneimine unit has a molecular weight in the range of about 200 Daltons to about 25,000 Daltons.

4. The composition of claim 1, wherein the recurring backbone degradable unit is a recurring unit of Formula (I): 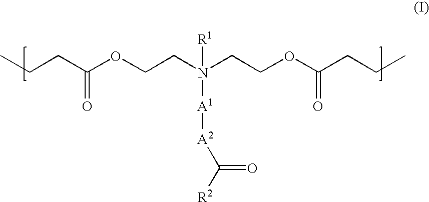
wherein:
A¹ is absent or an optionally substituted substituent selected from the group consisting of: alkyl, alkenyl, alkynyl, heteroalkyl, heteroalkenyl, heteroalkynyl and —(CH₂)ₙ₁-D-(CH₂)ₙ₂—;
wherein n1 and n2 are each independently 0 or an integer in the range of 1 to 10; and
D is an optionally substituted substituent selected from the group consisting of cycloalkyl, cycloalkenyl, cycloalkynyl, aryl, heteroaryl and heterocyclyl;
A² is absent, an oxygen atom or —N(R^{N}), wherein R^{N} is H or C₁₋₆ alkyl;
R¹ is an electron pair, hydrogen, or an optionally substituted substituent selected from the group consisting of alkyl, alkenyl, alkynyl, heteroalkyl, heteroalkenyl, heteroalkynyl, aryl, heteroaryl, and heterocyclyl,
wherein if R¹ is hydrogen, or an optionally substituted substituent selected from the group consisting of alkyl, alkenyl, alkynyl, heteroalkyl, heteroalkenyl, heteroalkynyl, aryl, heteroaryl, and heterocyclyl, then the nitrogen atom to which R¹ is attached has a positive charge; and
R² is selected from the group consisting of C₂-C₅₀ alkyl, C₂-C₅₀ heteroalkyl, C₂-C₅₀ alkenyl, C₂-C₅₀ heteroalkenyl, C₂-C₅₀ alkynyl, C₂-C₅₀ heteroalkynyl, C₅-C₅₀ aryl, C₅-C₅₀ heteroaryl, —(CH₂)ₚ₁-E-(CH₂)ₚ₂—, and sterol;
wherein p1 and p2 are each independently 0 or an integer in the range of 1 to 40; and
E is an optionally substituted substituent selected from the group consisting of cycloalkyl, cycloalkenyl, cycloalkynyl, aryl, heteroaryl and heterocyclyl.

5. The composition of claim 4, wherein R² is selected from the group consisting of oleyl, lauryl, myristyl, palmityl, margaryl, stearyl, arachidyl, behenyl, lignoceryl and a sterol.

6. The composition of claim 1, wherein the recurring backbone degradable unit is:

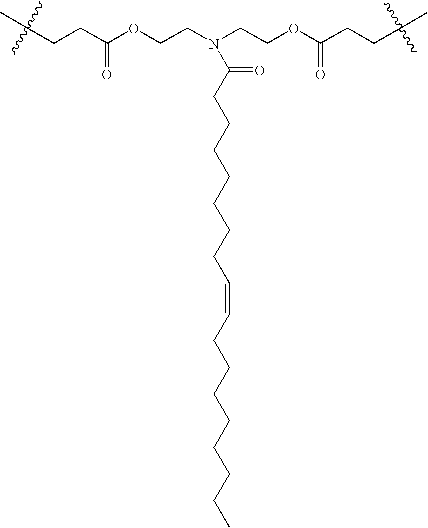

7. The composition of claim 6, wherein the recurring backbone PEI unit has a molecular weight of about 1200 Daltons.

8. The composition of claim 6, wherein the recurring backbone PEI unit is a branched PEI unit.

9. The composition of claim 8, wherein the recurring backbone PEG unit has a molecular weight of about 454 Daltons.

10. The composition of claim 1, wherein the water soluble degradable crosslinked cationic polymer comprises about 1 mole % to about 95 mole % of recurring backbone degradable unit based on the total moles of recurring units in the water soluble degradable crosslinked cationic polymer.

11. The composition of claim 1, wherein the water soluble degradable crosslinked cationic polymer comprises about 1 mole % to about 35 mole % of the recurring backbone cationic polyethyleneimine unit based on the total moles of recurring units in the water soluble degradable crosslinked cationic polymer.

12. The composition of claim 1, wherein the water soluble degradable crosslinked cationic polymer comprises about 1 mole % to about 80 mole % of the recurring backbone polyethylene glycol unit based on the total moles of recurring units in the water soluble degradable crosslinked cationic polymer.

13. A method of making the water soluble degradable crosslinked cationic polymer of claim 1, comprising:
dissolving a first reactant comprising recurring ethyleneimine units in an organic solvent to form a dissolved or partially dissolved polymeric reactant;
reacting the dissolved or partially dissolved polymeric reactant with a degradable monomeric reactant to form a degradable crosslinked polymer, wherein the degradable monomeric reactant comprises a lipid group; and
reacting the degradable crosslinked polymer with a third reactant, wherein the third reactant comprises recurring polyethylene glycol units.

14. The method of claim 13, wherein the first reactant is polyethyleneimine

15. The method of claim 13, wherein the degradable monomeric reactant is a compound of Formula (II): 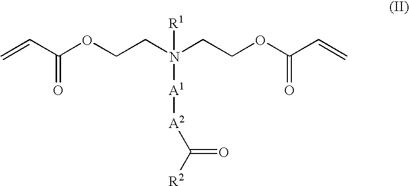
wherein:
A¹ is absent or an optionally substituted substituent selected from the group consisting of: alkyl, alkenyl, alkynyl, heteroalkyl, heteroalkenyl, heteroalkynyl and —(CH₂)ₙ₁-D-(CH₂)ₙ₂—;
wherein n1 and n2 are each independently 0 or an integer in the range of 1 to 10; and
D is an optionally substituted substituent selected from the group consisting of cycloalkyl, cycloalkenyl, cycloalkynyl, aryl, heteroaryl and heterocyclyl;
A² is absent, an oxygen atom or —N(R^{N}), wherein R^{N} is H or C₁₋₆ alkyl;
R¹ is an electron pair, hydrogen, or an optionally substituted substituent selected from the group consisting of alkyl, alkenyl, alkynyl, heteroalkyl, heteroalkenyl, heteroalkynyl, aryl, heteroaryl, and heterocyclyl,
wherein if R¹ is hydrogen, or an optionally substituted substituent selected from the group consisting of alkyl, alkenyl, alkynyl, heteroalkyl, heteroalkenyl, heteroalkynyl, aryl, heteroaryl, and heterocyclyl, then the nitrogen atom to which R¹ is attached has a positive charge; and
R² is selected from the group consisting of C₂-C₅₀ alkyl, C₂-C₅₀ heteroalkyl, C₂-C₅₀ alkenyl, C₂-C₅₀ heteroalkenyl, C₂-C₅₀ alkynyl, C₂-C₅₀ heteroalkynyl, C₅-C₅₀ aryl, C₅-C₅₀ heteroaryl, —(CH₂)ₚ₁-E-(CH₂)ₚ₂—, and sterol;
wherein p1 and p2 are each independently 0 or an integer in the range of 1 to 40; and
E is an optionally substituted substituent selected from the group consisting of cycloalkyl, cycloalkenyl, cycloalkynyl, aryl, heteroaryl and heterocyclyl.

16. The method of claim 15, wherein R² is selected from the group consisting of oleyl, lauryl, myristyl, palmityl, margaryl, stearyl, arachidyl, behenyl, lignoceryl and a sterol.

17. The method of claim 13, wherein the third reactant is polyethylene glycol or methoxypolyethylene glycol.

18. The method of claim 17, wherein the compound of Formula (II) and the PEI are present in a mole ratio in the range of about 0.1:1 to about 50:1, respectively.

19. The method of claim 17, wherein the PEG and the PEI are present in a mole ratio in the range of about 0.1:1 to about 12:1, respectively.

20. A method of delivering short interfering RNA (siRNA) into a cell comprising:
combining the water soluble degradable crosslinked cationic polymer of claim 1 with the siRNA to form a mixture; and
contacting one or more cells with the mixture.

21. The method of claim 20, wherein the siRNA has 19 to 27 base pairs.

22. The method of claim 20, wherein the cells are mammalian cells.

23. The method of claim 22, wherein the mammalian cells are cancer cells.

24. The method of claim 22, wherein the siRNA is an siRNA corresponding to at least a portion of a coding region of a lipoprotein gene segment.

25. The method of claim 24, wherein the lipoprotein is apolipoprotein-B.

26. A method of treating or reducing the risk of cardiovascular disease comprising administering a therapeutically effective amount of an siRNA corresponding to at least a portion of a coding region of a lipoprotein gene segment complexed with the water soluble degradable crosslinked cationic polymer of claim 1.

27. The method of claim 26, wherein the lipoprotein is apolipoprotein-B.

28. A device for transfecting a eukaryotic cell with siRNA comprising a solid surface at least partially affixed with a composition comprising a transfection agent, wherein the transfection reagent is selected from the group consisting of a water soluble degradable crosslinked cationic polymer, cationic polymer, lipopolymer, pegylated cationic polymer, pegylated lipopolymer, cationic lipid, pegylated cationic lipid, and cationic degradable pegylated lipopolymer.

29. The device of claim 28, wherein the solid surface is a dish bottom, a multi-well plate, a continuous surface, a bead, a fiber, or a pellet.

30. The device of claim 28, wherein the solid surface is a polystyrene resin, epoxy resin, natural resin, glass, or metal.

31. The device of claim 28, wherein the transfection reagent is affixed on the surface by evenly spreading the reagent on the solid surface or spotting the transfection reagent on the solid surface manually or by an automated mechanism.

32. The device of claim 28, wherein the transfection agent is a water soluble degradable crosslinked cationic polymer.

33. The device of claim 32, wherein the water soluble degradable crosslinked cationic polymer comprises:
(a) a recurring backbone polyethylene glycol (PEG) unit,
(b) a recurring backbone cationic polyethyleneimine (PEI) unit, and
(c) a recurring backbone degradable unit that comprises a side chain lipid group.

34. The device of claim 33, wherein the water soluble degradable crosslinked cationic polymer comprises a recurring backbone PEI unit having a molecular weight of about 1200 Daltons, a recurring backbone PEG unit having a molecular weight of about 454 Daltons and a recurring backbone degradable unit which is:

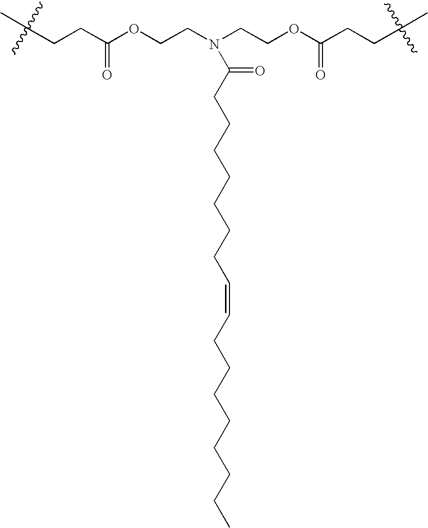

35. The device of claim 33, wherein the molecular weight of the water soluble degradable crosslinked cationic polymer is in the range of about 500 Daltons to about 1,000,000 Daltons.

36. The device of claim 33, wherein the molecular weight of the water soluble degradable crosslinked cationic polymer is in the range of about 2000 Daltons to about 200,000 Daltons.

37. A method for introducing siRNA into eukaryotic cells comprising:
(a) providing a solid surface at least partially coated with the water soluble degradable crosslinked cationic polymer of claim 1;
(b) adding the siRNA to be introduced into the eukaryotic cells onto the cell surface; and
(c) seeding cells on the solid surface at a sufficient density and under appropriate conditions for introduction of siRNA into the eukaryotic cells.

38. The method of claim 37, wherein the solid surface is selected from the group consisting of flasks, dishes, multi-well plates, glass slides and implanted devices.

39. The method of claim 37, wherein the water soluble degradable crosslinked cationic polymer is affixed on the surface by evenly spreading the reagent on the solid surface or spotting the transfection reagent on the solid surface manually or by an automated mechanism.

40. The method of claim 37, wherein the eukaryotic cells are mammalian cells.

41. The method of claim 37, further comprising:
(d) detecting whether the siRNA have entered the cells.
